(19) United States
(12) Patent Application Publication
(10) Pub. No.: US 2025/0009277 A1
(43) Pub. Date: Jan. 9, 2025

(54) CARDIAC DIAGNOSTIC SYSTEM

(71) Applicant: Cardathea, Inc, Waban, MA (US)

(72) Inventors: Dwight MEGLAN, Westwood, MA (US); Ryan Scott BARDSLEY, Manchester, MA (US); Richard BERENSON, Waban, MA (US); Brian H. CRAIG, Minneapolis, MN (US)

(21) Appl. No.: 18/758,662

(22) Filed: Jun. 28, 2024

Related U.S. Application Data
(60) Provisional application No. 63/511,783, filed on Jul. 3, 2023.

(51) Int. Cl. A61B 5/367; A61B 5/00; A61B 5/06; A61B 5/287; A61B 5/308; A61B 5/339; A61B 5/36

(52) U.S. Cl. CPC A61B 5/367; A61B 5/065; A61B 5/287; A61B 5/308; A61B 5/339; A61B 5/36; A61B 5/6852

(57) ABSTRACT

Provided herein are systems, devices, and methods for performing a cardiac diagnostic procedure. A system for locating segments of a cardiac conduction pathway in a patient includes: a plurality of sensors placed on the surface of the body of the patient, a data collection system for collecting sensor data from the plurality of sensors; a data processing system for calculating CEA data from the sensor data; and a display system for presenting a three-dimensional model of the location of the segments of the cardiac conduction pathway based on the calculated CEA data.

### CROSS-REFERENCE TO RELATED APPLICATION

This application claims priority to U.S. Provisional Application No. 63/511,783, filed Jul. 3, 2023, the entirety of which is incorporated herein by reference.

### FIELD

The embodiments disclosed herein relate generally to methods, systems, and devices for (1) imaging an organic conduction pathway, such as a cardiac conduction pathway; (2) navigating a therapy device to such an organic conduction pathway; and (3) determining whether a therapy device has engaged an organic conduction system pathway.

### BACKGROUND

Normal heart function relies on delivering contraction-triggering electrical impulses to cardiomyocytes in a well-defined spatiotemporal pattern known as “sinus rhythm.” This pattern is maintained by the cardiac conduction pathway (sometimes referred to herein as “conduction system”), as shown in FIG. 1. An electrical signal propagating through the cardiac conduction pathway initiates in the Sinoatrial (“SA”) node, and then travels around the right atrium to the left atrium and to the Atrioventricular (“AV”) node, where it pauses before propagating through the His Bundle, then through the left bundle branch (“LBB”) and the right bundle branch (“RBB”)-both of which are located in the septum between the ventricles, and then through the branching Purkinje fibers, before spreading across the ventricles. This electrical activity in the heart produces an electric field that can be detected, such as by skin surface electrodes in a pattern called an electrocardiogram (“ECG”), as shown in FIG. 2. An ECG shows how the electrical field strength fluctuates during each heartbeat. When detected, for example, as the difference between the voltages detected on a left arm electrode and a right arm electrode, the electric field produced in a single cardiac beat that shows a characteristic pattern of “waves” that are labeled/named by a letter P, Q, R, S, and T. Each wave spans a section of the ECG pattern that has a peak. It is common for both the section and the peak to be referred to by the same letter. The P section or “P wave” corresponds to the propagation of electrical activity across the surface of the atria. The Q-R-S complex comprises the Q, R, and S waves and corresponds to the propagation of electrical activity across the surface of the ventricles. The T wave corresponds to the repolarization of the ventricles.

Complementing the waves are sections of the cardiac cycle called “segments” that start at the end of one wave and end at the beginning of another. For example, a PR segment starts at the end of the P wave and ends just before the beginning of the Q wave (FIG. 2). It is also sometimes useful to divide the cardiac cycle into sections called “intervals.” Some intervals start at the peak of one wave and end at the peak of another. For example, an RR interval starts at the peak of the R wave of a first cardiac beat and ends at the peak of an R wave of a subsequent cardiac beat. Some intervals start at the beginning of the wave and end at the beginning of the next wave. For example, the PR interval starts at the beginning of the P wave and ends at the beginning of the Q wave.

Disease may disrupt the cardiac conduction pathway and, thus, the sinus rhythm, leading to reduced cardiac output, morbidity, and mortality associated with reduced oxygen delivery to the body. For example, disruptions in this conduction pathway can cause irregular heartbeats (“arrhythmias”) that, if left untreated, can reduce heart output and lead to severe conditions such as heart failure. Arrhythmias, such as tachycardia, affect heart rate. The most common types of tachycardia (e.g., heartbeats greater than 100 bpm) are atrial fibrillation (“AF”) and ventricular tachycardia (“VT”). Another type of arrhythmia, Bradycardia (e.g., heartbeats less than 50 bpm), can be caused by sinus node dysfunction or AV block.

To mitigate arrhythmias, many patients receive implantable devices for pacing the electrical system of the heart to induce sinus rhythm. For example, implantable cardiac defibrillators (“ICD”s) may be used to prevent tachycardia, while pacemakers (“PPM”s) may be used to prevent bradycardias (and sometimes for tachycardias). Pacing leads (implantable electrical leads, or wires, containing one or more electrodes) for these devices are wired into at least one of three locations in the heart to deliver precisely timed electrical impulses. The locations are typically the right atrium (“RA”), the right ventricle (“RV”), and sometimes the left ventricle (“LV”). The electrical impulses “pace” the cardiac electrical system and restore the heart to a normal sinus rhythm.

Pacing leads for PPMs and ICDs are typically delivered to the heart percutaneously through the neck. A clinician guides the pacing lead through a patient's vein to the target using fluoroscopy. Typically, most ventricular leads are placed in traditional target locations, such as the lower septum, ventricular apex, and/or ventricular free wall of the heart because these placements are generally easy to achieve. That is, clinicians may quickly navigate to these targets with conventional scanning techniques (e.g., fluoroscopy, echocardiography, MRI/CT images, etc.). However, long-term follow-up studies have shown that these conventional placements can lead to ventricular desynchrony, myocardial perfusion defects, heart failure, and/or additional arrhythmias.

Alternatively, “conduction system pacing”, also referred to as “physiologic pacing”, exploits the working portions of the cardiac conduction pathway to restore a substantially normal electrical propagation, and thus, restore the heart to a more normal rhythm. Conduction system pacing improves ventricular synchronization and increases cardiac output relative to pacing at other locations of the heart. Ideal targets for conduction system pacing (“CSP”) are the His Bundle, the LBB, and/or the RBB.

Unfortunately, reaching these pacing lead targets can be challenging. In particular, endocardial placements are difficult because the septum and the bundles cannot be directly and/or timely detected using conventional means such as fluoroscopy or echocardiography (which are the primary methods of imaging tissue inside a patient during a surgical procedure). For example, fluoroscopy cannot detect the cardiac conduction system or directly visualize the septum; echocardiography has too low a resolution to distinguish the septum from the rest of the heart and cannot detect the cardiac conduction pathway. Similarly, MRI/CT imaging of the cardiac conduction system requires contrast dye, has only been done ex-vivo, cannot be done in real-time, and is rarely available in a catheterization lab (also referred to as a “cath lab”). Consequently, although considered to be a preferred lead placement configuration, conduction system lead placement today is a trial-and-error effort, requires extended procedure times, and is typically achieved only by the most highly skilled electrophysiologists. For instance, conduction system pacing reported a 27% increase in procedure time and a 39% increase in fluoroscopy time compared to right ventricular pacing.

Additionally, other existing cardiac mapping systems, generally, cannot directly visualize the cardiac conductive system and/or septum in real-time or with enough accuracy for guiding and attaching pacing leads to target locations for conduction system pacing. For example, electroanatomic mapping (“EAM”) systems are not designed to identify the passage of concentrated electrical signals through the His Bundle, the LBB, the RBB, and the Purkinje fibers. Instead, commercially available EAM systems are optimized for creating tissue maps and visualizing wavefront propagation across the atrial and ventricular walls. This method of non-fluoroscopic mapping is typically based on using an activation sequence to track and localize the tip of a mapping catheter in conjunction with electrical activity recorded by the catheter. The standard approach to developing such a map can take an hour or more and requires repeatedly placing a sensing electrode on different spots of the heart to collect data over several cardiac cycles. This extended time to map a patient's heart (as compared to conventional navigating methods) adds considerable cost to the procedure and may be detrimental to the patient who may not be well enough to withstand such an extended procedure. Another EAM technique employs a non-contact 64-electrode basket catheter placed inside the heart to map multiple points inside the ventricle. However, mapping a patient's heart using such techniques cannot be realized in real time and/or with enough precision for conduction system pacing. These techniques are optimized to locate the broad wavefront that propagates over the surface of the ventricle by interpolating the wavefront from the timing of signals received at, or calculated for, different points along the surface. Because this type of interpolation does not work well for mapping a narrow path that may be missed due to the placement of the sensing electrodes, some clinicians (also referred to as “implanters” herein) have resorted to doing detailed, time-consuming, multi-electrode maps of the crest of the intraventricular septum.

Another approach to EAM, often called iEAM, utilizes an inverse algorithm to reconstruct cardiac electrical activity from recorded body surface potentials. These potentials are collected from a large number (e.g., 64 or 182) of body surface electrodes (the three-dimensional (“3D”) location of which must be known with reasonable precision) and projected onto a patient-specific thorax and heart model derived from MRI or CT data. However, even iEAM maps are not precise enough to accurately locate the His Bundle, the LBB, the RBB, and/or other segments of the cardiac conduction system. Consequently, the EAM/IEAM approach has been used to help pre-plan ventricular tachycardia, atrial flutter, and atrial tachycardia ablation procedures, but not for real-time visualization during a surgical procedure.

Other techniques for diagnosing cardiac dysfunctions include Vector cardiography (“VCG”) and “CineECG”. VCG is calculated from ECG signals received by electrodes precisely located at specific, pre-defined anatomic locations on the patient's body. The strength of each signal is used to weigh a summation of the vectors from the heart center to the electrodes, producing a moving vector with a base at the heart center. The tip of this moving vector traces a space curve that may be used to detect an acute myocardial infarction, right ventricular hypertrophy, and Tawar arm blockade. The CineECG, integrates the VCG over time to produce an estimate of the movement of the average location of electrical activation, also called the mean temporo-spatial isochrone (“iTSI”).

These current approaches typically require that MRI or CT data be collected and integrated into the analysis system in order to produce useful data. None of these approaches produce a map of the septum, the His Bundle, the LBB, the RBB, the Purkinje fibers and/or other central segments of the cardiac conduction system/pathway sufficiently accurately and/or timely to provide real-time guidance of a pacing lead to an endocardial target. Consequently, conduction system pacing cannot be easily achieved through use of these EAM approaches.

For these and other reasons, there is a need for improved systems, devices, and methods for accurately imaging the septum, the His Bundle, the RBB, the LBB, and/or other segments of an electrical conduction system in real-time for diagnosing a condition of the heart and/or guiding an instrument and/or lead to an endocardial target.

### SUMMARY

According to an aspect of the present disclosure, the techniques described herein relate to a system for mapping at least a portion of a cardiac conduction pathway in a patient, the system including: a plurality of sensors; a data collection system for collecting sensor data from the plurality of sensors; a data processing system for calculating a center of electrical activity (CEA) data from the sensor data; and a display system for presenting a three-dimensional (3D) model of a portion of the cardiac conduction pathway based on the calculated CEA data.

In some aspects, the techniques described herein relate to a system, wherein the CEA data is determined by calculating a single equivalent dipole (SED).

In some aspects, the techniques described herein relate to a system, wherein the 3D model of the portion the cardiac conduction pathway is displayed with respect to images of a gross anatomy of a heart.

In some aspects, the techniques described herein relate to a system, wherein the plurality of sensors detect a signal propagating through segments of the cardiac conduction pathway during a PR segment of a cardiac cycle.

In some aspects, the techniques described herein relate to a system, wherein the display system displays the portion of the cardiac conduction pathway located between an atrioventricular (AV) node and Purkinje fibers of a heart.

In some aspects, the techniques described herein relate to a system, wherein the data collecting system includes a digital converter for generating high resolution data from very low voltage signals sensed by the plurality of sensors.

In some aspects, the techniques described herein relate to a system, wherein the voltage signals are below 0.1 mV.

In some aspects, the techniques described herein relate to a system, wherein the data processing system enhances the sensor data via one or more of a low pass filter; a high pass filter; common mode reduction; and/or differentially weighting data from different sensors of the plurality of sensors.

In some aspects, the techniques described herein relate to a system, further including a sensor location system for identifying a location of each sensor of the plurality of sensors in a 3D space.

In some aspects, the techniques described herein relate to a system, wherein the sensor location system includes a scanner, a CT machine, and/or an MRI machine configured to generate a 3D image.

In some aspects, the techniques described herein relate to a system, wherein a machine learning algorithm trained to identify sensors in a 3D image identifies and locates the plurality of sensors in the 3D image generated by the sensor location system.

In some aspects, the techniques described herein relate to a system, further including a garment and/or a harness for receiving the plurality of sensors.

In some aspects, the techniques described herein relate to a system, wherein the data collection system is configured to indicate to a user whether a particular sensor of the plurality of sensors is improperly positioned and/or malfunctioning.

In some aspects, the techniques described herein relate to a system, wherein the data collection system is configured to cause one or more sensors of the plurality of sensors to transmit one or more signals.

In some aspects, the techniques described herein relate to a system, wherein the system is configured to filter the sensor data by applying a different broad band pass filter and/or narrow band pass filter to selected frequencies.

In some aspects, the techniques described herein relate to a system, wherein the selected frequencies are between about 0.5 and 55 Hz or between about 65 and 300 Hz.

In some aspects, the techniques described herein relate to a system, wherein the data processing system is configured to remove CEA data corresponding to timepoints for which a voltage at a particular timepoint of the timepoints is below a threshold value.

In some aspects, the techniques described herein relate to a system, wherein each sensor of the plurality of sensors is weighted when determining a CEA based on a voltage-drop across at least one chord between sensors of the plurality of sensors.

In some aspects, the techniques described herein relate to a system, wherein a location of a portion of the cardiac conduction pathway is determined by combining CEA data from multiple cardiac cycles.

In some aspects, the techniques described herein relate to a system, wherein the combined CEA data from multiple cardiac cycles accounts for discrepancies due to motion of a heart during each cardiac cycle.

In some aspects, the techniques described herein relate to a system, wherein the combined CEA data includes combining the CEA data from the multiple cardiac cycles into a best fit a model.

In some aspects, the techniques described herein relate to a system, wherein the system is further configured to determine a location of a septum of a patient's heart using data collected from an emitting device.

In some aspects, the techniques described herein relate to a system, wherein the location of the septum constrains the best fit model of the cardiac conduction pathway.

In some aspects, the techniques described herein relate to a system, wherein the system is used for navigating a catheter to a target on the septum.

In some aspects, the techniques described herein relate to a system wherein a probability distribution for the location of the cardiac conduction pathway is graphically displayed on an image of the septum.

In some aspects, the techniques described herein relate to a system, further including an emitting device, wherein the data processing system is configured to determine a location of the emitting device relative to the location of a portion of the cardiac conduction pathway.

In some aspects, the techniques described herein relate to a system, further including a control device connected to a proximal end of the emitting device.

In some aspects, the techniques described herein relate to a system, wherein the control device activates and/or controls a signal emitted by the emitting device.

In some aspects, the techniques described herein relate to a system, wherein the control device controls movement of the emitting device.

In some aspects, the techniques described herein relate to a system, further including an emitting device, wherein the data processing system is configured to determine an orientation of the emitting device relative to a location of the portion of the cardiac conduction pathway.

In some aspects, the techniques described herein relate to a system, wherein a location of the portion of the cardiac conduction pathway is determined before and after a therapy is applied to the patient.

In some aspects, the techniques described herein relate to a system, wherein the data processing system enhances the sensor data by selecting a filter based on whether or not an implanted pacing lead has recently delivered a pacing signal.

In some aspects, the techniques described herein relate to a system where the system is also used for navigating a catheter to a target in a heart.

In some aspects, the techniques described herein relate to a system, wherein the system can also be used to develop a pacing strategy for a patient.

In some aspects, the techniques described herein relate to a system, wherein the system can also be used to determine whether conduction system pacing has been achieved.

In some aspects, the techniques described herein relate to a method of mapping at least a portion of a cardiac conduction pathway including: sensing, via sensors, signals indicative of cardiac electrical signals propagating through the cardiac conduction pathway; combining the signals from each sensor, via a data collection system, to generate a first data stream; identifying, via a data processing system, a waveform from the data stream via a low resolution sampling of the first data stream; sampling, via the data processing system, a segment of the identified waveform of the first data stream at a high-resolution to generate a second data stream; determining, via the data processing system, center of electrical activity (CEA) data based on the second data stream; and generating, via a display system, a three-dimensional (3D) model of the CEA data in real time, wherein the 3D model is indicative of the cardiac conduction pathway.

In some aspects, the techniques described herein relate to a method, wherein determining the CEA data includes performing single equivalent dipole (SED) analysis of the second data stream.

In some aspects, the techniques described herein relate to a method, further including generating a real time image of a heart and overlaying the 3D model with reference to the real time image of the heart.

In some aspects, the techniques described herein relate to a method, wherein the identified waveform is an electrocardiogram including a P wave, QRS complex and T wave.

In some aspects, the techniques described herein relate to a method, wherein the segment of the identified waveform is a PR segment of the identified waveform.

In some aspects, the techniques described herein relate to a method, further including determining locations of the sensors with respect to a heart of a subject.

In some aspects, the techniques described herein relate to a method, wherein determining locations of the sensors includes scanning the subject and the sensors, wherein the sensors are disposed on a body of the subject.

In some aspects, the techniques described herein relate to a method, wherein determining CEA data is further based on the determined locations of the sensors.

In some aspects, the techniques described herein relate to a method, further including sensing, via the sensors, a single dipole signal from an emitting device disposed within a heart of a subject.

In some aspects, the techniques described herein relate to a method, further including determining, via the processing system, a location and orientation of the emitting device using SED analysis.

In some aspects, the techniques described herein relate to a method, further including generating, via the display system, a 3D representation of the emitting device with respect to the 3D model indicative of the cardiac conduction pathway, wherein the 3D representation indicates a location and orientation of the emitting device in real time.

In some aspects, the techniques described herein relate to a method, further including navigating the emitting device so that it is proximate to a desired portion of the cardiac conduction pathway based on the 3D representation.

In some aspects, the techniques described herein relate to a method, further including detecting an electrode has been placed so as to capture proximal to the cardiac conduction pathway.

The details of one or more aspects of the disclosure are plurality forth in the accompanying drawings and the description below. Other features, objects, and advantages of the techniques described in this disclosure will be apparent from the description and drawings, and from the claims.

### BRIEF DESCRIPTION OF THE DRAWINGS

The foregoing and other features and advantages of the present disclosure will be apparent from the following description of embodiments hereof as illustrated in the accompanying drawings. The accompanying drawings, which are incorporated herein and form a part of the specification, further explain the principles of the present disclosure and are meant to enable a person skilled in the pertinent art to make and use the embodiments of the present disclosure. The drawings are not to scale.

FIG. 1 illustrates an anatomic sectional view of a mammalian heart.

FIG. 2 illustrates a graph of an ECG signal of a mammalian patient.

FIG. 3 illustrates a schematic view of a system for performing a medical procedure, according to an embodiment.

FIG. 4A is an image of an array of sensors attached to a porcine subject derived from a digital 3D scan of that subject.

FIG. 4B depicts front and back views of an array of sensors attached to a human subject, according to an embodiment.

FIG. 5 is a flow chart illustrating a method for collecting data, according to an embodiment.

FIG. 6 is a flow chart illustrating a method of processing a data stream, according to an embodiment.

FIG. 7 is a flow chart illustrating a method of data processing, according to an embodiment.

FIG. 8 is a flow chart illustrating a method of data collection and processing, according to an embodiment.

FIGS. 9A and 9B are plots of data obtained from one or more cardiac cycles, according to an embodiment.

FIG. 9C depicts a space curve graphical representation of a section of a cardiac conduction pathway, according to an embodiment.

FIGS. 10A-10C depict space curve graphical representations illustrating propagation of a signal along a segment of a cardiac conduction pathway, according to an embodiment.

FIG. 11 is a hardware block diagram of a computing device according to an embodiment.

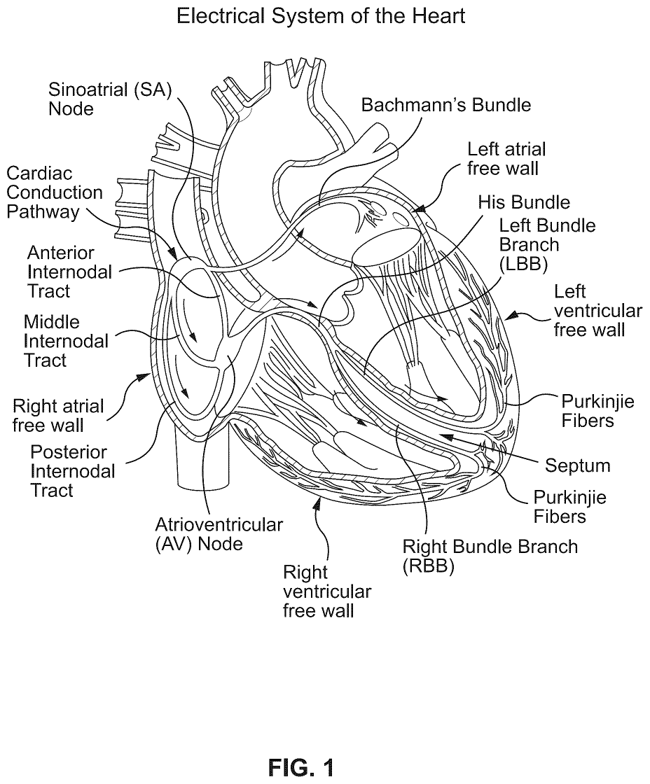

### DETAILED DESCRIPTION

Reference will now be made in detail to the present embodiments of the technology, examples of which are illustrated in the accompanying drawings. Like reference numerals have been used to identify like elements throughout this disclosure. The following description is not intended to limit the present disclosure to particular embodiments, and it should be construed as including various modifications, equivalents, and/or alternatives of the embodiments described herein.

The techniques presented herein directly visualize a cardiac conduction system pathway in real time for diagnostic and/or surgical procedures. For example, the techniques presented herein provide target locations for conduction system pacing (sometimes referred to as “physiologic pacing”) and guide an implanter to these locations. These techniques enable a more diverse group of clinicians to complete a procedure quickly and accurately with improved efficacy for a subject (sometimes referred to herein as a “patient”). In particular, the techniques presented herein provide real-time, or near real-time (e.g., within 100 milliseconds (“ms”)) 3D mapping of the cardiac conduction system, such as the SA node, the AV node, the His Bundle, the LBB, and/or the RBB, for real-time visualization of and navigation or guidance to a target location within the heart (e.g., on the septum). Accordingly, a clinician may quickly and easily navigate to the septum (as compared to conventional techniques) and attach pacing leads for conduction system pacing (“CSP”) at a desired location thereon.

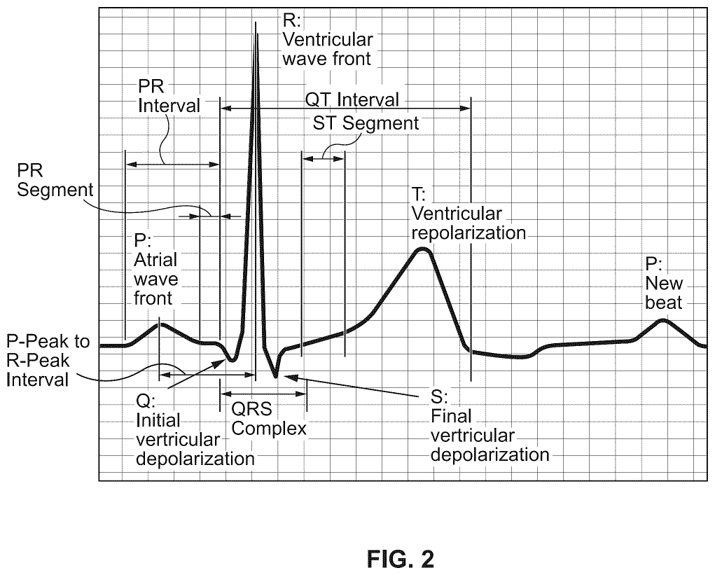

For example, the techniques presented herein include locating a moving center of electrical activation (“MCEA”) within the cardiac conduction system using Single Equivalent Moving Dipole (“SEMD”) analysis. The MCEA may be representative of an electrical signal as it travels along the cardiac conduction pathway (e.g., from the SA node, through the AV node, the His Bundle, the LBB, the RBB and ultimately through the Purkinje fibers). The SEMD analysis of electrocardiographic data is a method for analyzing the pattern of electrical signals emanating from the heart (e.g., the cardiac conduction system) and arriving on body-surface electrodes. The method estimates the location (in the form of 3D coordinates) and the moment (in the form of a 3D) direction vector) of a single equivalent dipole (“SED”) of an electrical signal as it propagates along the cardiac conduction pathway over a series of time points corresponding to an ECG waveform. In particular, the series of time points correspond to a desired interval or segment, for example, a P-Peak to R-Peak interval of the ECG waveform, and in some embodiments, the series of time points may span the PR segment (see FIG. 2). The P-peak to R-peak interval starts at the peak of the P wave and ends at the peak of the R wave. In some implementations, a desired segment for SEMD analysis may be the PR segment. The PR segment may begin at the end of the P wave and end at the beginning of the Q wave or QRS complex. In instances where the Q wave is not detected, the desired segment may extend from the end of the P wave to the beginning of the R wave.

In addition, the SEMD data may also be used to localize the site of origin in the heart of the abnormal electrical excitation causing VT. For example, a myocardial scar due to an infarct can retain a strand of electrically active cells that allows a wave of activation passing across the ventricles to come back and stimulate a new wave of activation—causing an extra “beat.” The “origin” of the arrhythmia is where such an aberrant pathway (also called a “reentrant circuit”) exits the scar. The SEMD, when based on high-time- and data-resolution data can uncover the location of this “origin.” If the origin re-excites the ventricle after the primary wave of electrical activity (e.g., the QRS complex) has passed (i.e., during the low-voltage ST segment), such location is revealed directly by the location of the SEMD. Otherwise, when the signal from the reentrance is overwhelmed by the signal from the QRS complex or the T wave, the location of the origin can be determined by applying a supra-threshold electrical stimulus to the heart at the same rate as the ventricular tachycardia using catheters with electrode tips, then looking for changes in the path and moment-orientation of the high-resolution-data-based SEMD.

SEMD can also be used to detect the location and orientation (relative to cardiac conduction pathways) of an emitter (e.g., an electrode) that has been placed near or inside the heart: a dipole signal emitted from a bipolar ablation electrode can be collocated in an “image space” with the SEMD data, enabling, for example, an ablation catheter to be navigated to the tachycardia's origin on the epicardial or endocardial surface of the free wall of the ventricles so that that origin can be ablated. Similarly, if a dipole signal is emitted by a pacing lead or other sensor/electrode proximate to the pacing lead (for example, on a catheter being used to place that pacing lead), then the location and/or orientation of the pacing lead can be collocated with the MCAD (e.g. SEMD) of the cardiac conduction system (e.g., the 3D plot of the His Bundle and/or the LBB and/or the RBB).

However, conventional systems, generally, cannot extract useful information from the PR segment (a segment of the ECG waveform starting at the end of the P wave and ending at the start of the Q wave or QRS complex), where the magnitude of the ECG signal is small relative to the noise level of that signal. Consequently, the signal from the PR segment is almost never examined or used, except occasionally to set an average isoelectric baseline as part of “normalizing” cardiac rhythm data across beats and/or as a baseline for beat noise estimation. Moreover, conventional systems using traditional SEMD analysis cannot accurately locate the AV node, the His Bundle, the LBB, the RBB, or the Purkinje fibers of the cardiac conduction pathway in real time using data from the low-magnitude signals produced during the PR segment.

Using the techniques presented herein, SEMD analysis of high-time-resolution data from the PR segment may determine, locate, and/or track the propagation of an MCEA along the AV node, the His Bundle, the LBB, the RBB, and the Purkinje fibers, and/or other portions of the cardiac conduction pathway. Generally, as used herein, the term “high-time-resolution” means a sampling rate greater than 500 Hz and the term “high-data-resolution” means greater than 16 bits of data per sample. The term “low-time-resolution” means a sampling rate less than or equal to 500 Hz and the term “low data-resolution” means less than or equal to 16 bits of data per sample.

Accordingly, techniques presented herein detect, locate (e.g., in 3D space), and display cardiac signals propagating from the AV node and through the His Bundle, the LBB, the RBB, and/or the Purkinje fibers, during the PR segment of a cardiac cycle in real-time (e.g., within 100 ms, 50 ms, and/or preferably within 10 ms from the PR segment). These techniques can be used on a variety of human and non-human patients. The PR segment of a cardiac waveform corresponds to a time period during which the wave front of cardiac electrical signal, or impulse, propagates from the AV node through the His Bundle, the LBB, the RBB, and the Purkinje fibers. According to at least one embodiment presented herein, the low-magnitude signals produced during the PR segment may be used to calculate an MCEA using SEMD analysis to locate the His Bundle, the LBB, the RBB, and Purkinje fibers of the cardiac conduction system, and/or portions thereof.

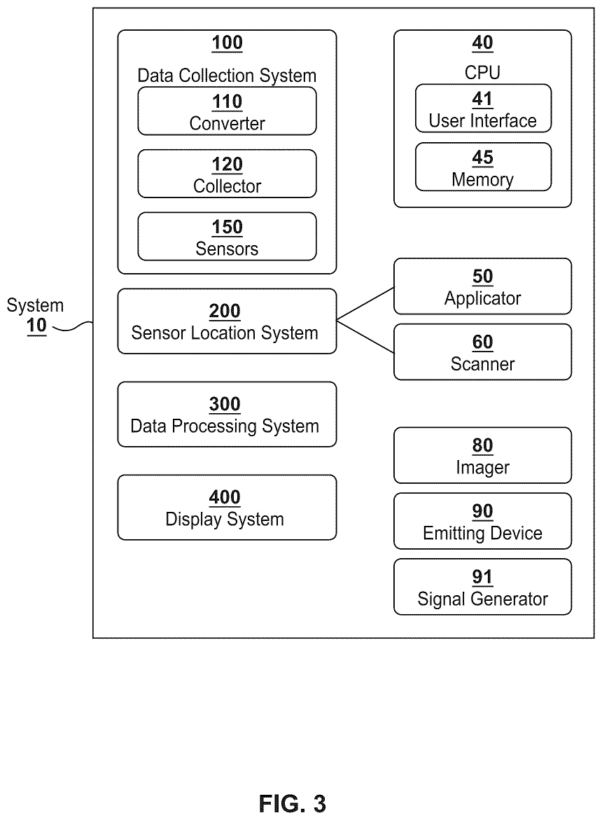

Referring now to FIG. 3, a schematic of a system 10 for detecting and displaying a conductive pathway within a mammal is illustrated, according to an embodiment. The system 10 includes a data collection system 100, a sensor location system 200, a data processing system 300, and a display system 400.

The data collection system 100 includes a converter 110, a collector 120, and one or more sensors 150. In some embodiments, the data collection system 100 comprises a Field Programmable Gate Arrays (“FPGA”) board. In some implementations, the data collection system 100 includes or communicates with a processor, such as a CPU 40 via wired communication and/or wireless communication (e.g., WiFi, Zigbee, NFC, Bluetooth, etc.).

The data collection system 100 receives and processes data (e.g., cleaning, sorting, prioritizing, filtering, removing outliers, and/or otherwise processing data) from signals transmitted by the sensors 150. The sensor location system 200 applies and locates the sensors 150 on an outer surface of a body or skin of a patient. The data processing system 300 processes data received from the data collection system 100 into MCEA data. The display system 400 generates and displays a 3D model of the MCEA data from the data processing system 300 and may include a graphical user interface (“GUI”). The GUI may include an input device (e.g., keyboard, mouse, joystick, foot pedal, microphone, touch pad, etc.) for receiving inputs from a user.

In the depicted embodiment, the converter 110 of the data collection system 100 converts the voltage of the electrical signal detected by each sensor 150 to a digital (e.g., binary) value. That is, the converter 110 may include one or more analog to digital (“A-D”) converters. The converter 110 samples voltages (e.g., indicative of cardiac voltage signals) from one or more sensors 150 at a rate that can be measured in Hertz (sample per second), converts the voltage signal into a digital data, and passes the data on to the collector 120.

In some implementations the converter 110 may include a plurality of converter components and/or devices configured to receive signals from the sensors 150. For example, each converter component may receive one or more signals from one or more of the sensors 150 and convert the one or more signals into digital data. In some implementations, converter 110 comprises one or more analog-to-digital converters that are of high data resolution, for example 12, 16, 20, 24, 36, or 48 bit resolution.

In some instances, the converter 110 may comprise one or more converters that are contained in “receiving modules” that are connected to the sensors 150 (e.g., one converter may be connected to between one and thirty-two or sixty-four sensors 150). That is, the receiving module can comprise multiple converters 110, each converter 110 may be electrically coupled or connected to at least one sensor 150 via a conductor (e.g., a wire). Each receiving module combines the outputs of the included converters 110 into a single partial data stream sent to the collector 120. In some implementations, the receiving modules of the data collection system 100 may be integrated into an applicator 50, discussed in detail below with reference to the sensor location system 200.

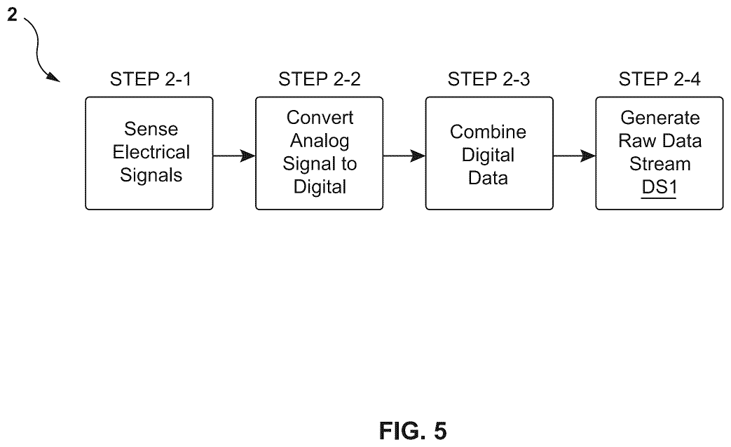

Regardless of the structure of the converter 110, the data from the converter 110 is transmitted to the collector 120 to generate a raw data stream. That is, the collector 120 assembles the data received or obtained from the converter 110 into a single stream of data (e.g., data stream DS1 of FIG. 5) for subsequent processing. In some implementations, the data stream may be saved to a storage device.

The sensors 150 can comprise one or more ECG sensors that include any of a wide variety of voltage-sensing devices. In some embodiments, the sensors 150 include one, two, or more magnetic field detectors. The sensors 150 may include one or more devices that can be passive and/or active in one or more ways, such as by being powered and/or by being configured to transmit and receive signals (e.g., direct current and/or alternating current signals). The sensors 150 may be capable of sensing voltages from the patient or subject, e.g., voltages from the subject's heart. The sensors 150 are configured to attach to the skin of the patient to electrically couple the sensor 150 to the subject's skin. For example, the sensors 150 may be affixed to subject's skin with an adhesive pad, and/or pierce the patient's skin surface. In some embodiments, one or more sensors 150 are concentrated in areas toward which SEMD moments are oriented during the PR segment. In some implementations, the sensors 150 are arranged radially about the torso and longitudinally between areas above and below the heart. In some instances, the sensors 150 are placed on the left arm, the right arm, left leg, and/or right leg, to provide a reference signal. In some embodiments, a pair of sensors 150 is placed on the patient's skin at locations between the fourth and fifth ribs on the left and/or right side of the sternum. The one or more sensors 150 are configured to electrically couple the converter 110, sense voltages from the subject's body, and transmit the sensed signals to the converter 110.

In some implementations, the data collection system 100 may be further configured to determine whether individual sensors 150 are functioning correctly, sometimes referred to herein as “sensor verification.” The sensors 150 may be connected to the data collection system 100 shortly before or after the sensors 150 are attached to the skin of the patient. The data collection system 100 can determine whether a signal is received from the sensor 150 and whether that signal is of high enough quality (e.g., above a predetermined threshold and/or clinician-adjustable threshold). For example, the data collection system 100 may determine that a signal that is weak, has a low signal-to-noise ratio, and/or does not reflect the pattern of cardiac electrical activity detected by one or more other sensors 150. The signal used for sensor verification can be generated by the heart and/or by one or more other sensors 150 transmitting one or more verification signals.

Still referring to FIG. 3, the system 10 may include one or more central processing units (“CPU”s) 40 (e.g., processors) and/or other computing components. The CPU 40 and/or other components of system 10 may include one or more electronic elements, electronic assemblies, and/or other electronic components, such as components selected from the group consisting of: memory storage components; analog-to-digital converters; rectification circuitry; state machines; microprocessors; microcontrollers; filters and other signal conditioners; sensor interface circuitry; transducer interface circuitry; and combinations thereof. The CPU 40 further includes a memory 45 that includes instructions for the system 10 to generate a 3D model of the cardiac conduction pathway. The instructions may further include guiding an instrument to an endocardial target with the generated 3D model. In some implementations, each of the data collection system 100, the sensor location system 200, the data processing system 300, and/or the display system 400 include either one or more CPUs 40 and/or are in communication with the CPU 40. In some instances, the memory 45 is external to and in communication with the one or more CPUs 40.

In the depicted embodiment, the system 10 further includes an imager 80 and an emitting device 90. The imager 80 (e.g., a fluoroscope, an MRI and/or CT device) images the interior of the patient. In some implementations, the interior of the patient may be imaged prior to or during a surgical procedure. The imaging data may be used for reference purposes during the generation of a 3D model of the cardiac conduction pathway. Additionally or alternatively, the imager 80 may be used during the surgical procedure.

The emitting device 90 (e.g., a catheter, electrode, and/or other device configured for insertion within the patient and for emitting an electrical signal) tracks a location of a pacing lead with respect to the cardiac conduction system target, and/or generally within the subject's heart and body. A proximal end of the emitting device 90 may include a control device for controlling movement of the emitting device 90 (e.g., a distal end of the emitting device) and/or control a signal emitted by the emitting device 90. In some embodiments, the emitting device 90 comprises an epicardial walker, endoscope, and/or a catheter, such as an electrophysiological sensing catheter, a lead-placement catheter, and/or another catheter for navigating the inside and/or outside of the heart. However, the emitting device 90 can be any device used in vivo to move around, within, and/or on the heart and can transmit an electrical signal through tissue from an internal location on and/or proximate to the heart of a patient.

In some embodiments, the distal end of the emitting device 90 comprises one or more electrodes configured to emit a monopolar, bipolar, or multipolar electric field. In some embodiments, each such electrode is electrically coupled with a signal generator 91. In some instances, the signal generator 91 is located near or is electrically coupled to the proximal end of the emitting device 90. In some embodiments, the emitting device can be configured to sense electrical fields in its vicinity. For example, the CPU 40 may instruct the signal generator 91 to turn off or change modes briefly (for example for 1, 10, or 100 ms or 1, 10 or 60 seconds) so that the electrodes become electrically coupled with the data collection system 100.

In some embodiments, the electrical signal transmitted by the emitting device 90 may be a 75 Hz, 90 Hz, 150 Hz, 200 Hz, 300, 500, 800, or 1,600 Hz sinusoidal signal or a combination of such sinusoidal signals. In some embodiments, the signal transmitted by device 90 can be fixed or variable, for example, at a frequency of 50 Hz, 65 Hz, 70 Hz, 75 Hz, 80 Hz, 90 Hz, 300 Hz, or 800 Hz. In some embodiments, a narrow bandpass filter (e.g., of the data processing system 300) can be set to isolate the signal transmitted by the emitting device 90. In some embodiments, the voltage level of the signal transmitted by the emitting device 90 is set to be at a level that does not affect the heartbeat of the patient. In some embodiments, such a safe voltage level of the emitting device 90 is determined for each individual subject.

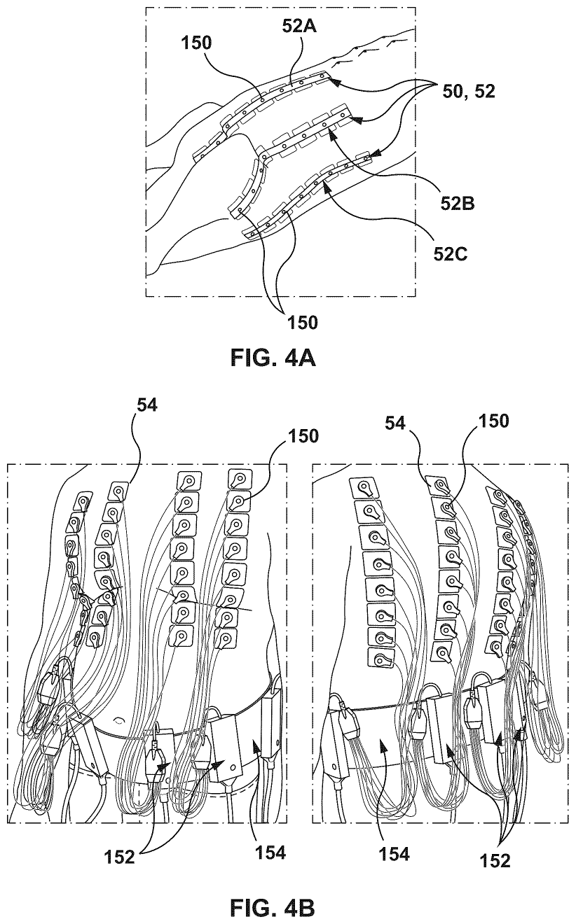

Now referring to FIGS. 4A and 4B, but with continued reference to FIG. 3, the plurality of sensors 150 are applied to a subject (e.g., a pig in FIG. 4A and a human torso in FIG. 4B) and the sensors' locations are determined with respect to the subject's body via the sensor location system 200. The sensor location system 200 may include an applicator 50 and/or a scanner 60. The applicator 50 may guide locations of and spacings between each of the sensors 150 on the patient's body. For example, the sensors 150 can be attached to and/or otherwise disposed on the patient's body using the applicator 50. The applicator 50 includes one or more rigid or flexible scaffolds that ensure the position of the sensors 150 relative to each other and the patient's body.

The applicator 50 may be one or more linear strips 52, as shown in the embodiment of FIG. 4A. In the depicted embodiment, the plurality of sensors 150 are distributed along the strips 52 and maintained in a desired position with respect to each other and the body. The strips 52 may have an adhesive surface for adhering to the subject's body. In some implementations, the strips may be glued or taped to the body. The strips may be formed by flexible polymer strips cut with regularly spaced holes. Buttons of snap electrodes (e.g., sensors 150) are pushed through the holes in the strip, and an adhesive-protecting tape is removed from the bottom of the sensors 150.

In an embodiment as depicted in FIG. 4A, a first sensor 150 on a first strip 52A is aligned with a pig's anterior collar bone and left shoulder to extend along a longitudinal axis of the pig's body. Once aligned, the first sensor 150 is adhered or otherwise attached to the subject's skin. The subsequent sensors 150 are placed on the strip and adhered or otherwise attached to the subject's body. Additional strips 52B, 52C with sensors 150 are similarly placed on the subject with the sensors 150 equally spread and symmetrically placed around a transverse circumference of the subject. Once the sensors 150 are arranged on and adhered to the body, the strips 52 may be removed. Consequently, the sensors 150 are coupled to and aligned along the pig's body in a desired arrangement. While only three strips 52A, 52B, and 52C are visible in FIG. 4A, in some embodiments, more than three strips 52 may be used. For example, eight strips 52 with sensors 150 may be radially aligned around the subject's body. In some implementations, a plurality of leads may connect each sensor 150 to one or more components of the data collection system 100, e.g., the converter 110, the collector 120, and/or subassemblies thereof.

In some embodiments, the applicator 50 guides the locations of one or more sensors 150 and patches 54 that may adhere the sensors 150 to a subject's skin as depicted in FIG. 4B. In the depicted embodiment, four snap electrodes (e.g., sensors 150) are substantially evenly spaced and aligned along a bottom of the subject's collar bone on the anterior side. Meanwhile, four evenly spaced sensors 150 are arranged across the top of the shoulder blades on the posterior side. Additional sensors 150 are longitudinally disposed below each of these top sensors 150. The applicator 50 may evenly space the additional electrodes along the longitudinal axis of the subject's body. In an embodiment as shown in FIG. 4B, eight vertical or longitudinally extending columns, having eight sensors 150 each, are arranged on the subject's body for a total of sixty-four sensors 150.

In some implementations, each column may include more than 8 sensors or less than 8 sensors. For example, each column may include 4, 6, 8, 10, 12, 14, or 16 sensors for a total 32, 48, 64, 80, 96, 112, or 128 sensors disposed on the subject. Additionally, or alternatively, more than 8 columns or less than 8 columns of sensors 150 may be disposed on the subject. In some implementations, applicator 50 may position sensors in a non-linear pattern, such as in a circular or square pattern.

In some implementations, the applicator 50 may include one or more guides that include markings to align the applicator 50 and the sensors 150 with one or more anatomical landmarks. For example, the applicator 50 may be a ruler, a tool, a scaffold, a guide garment, harness, and/or belt that can be worn over the torso and support placement of sensors 150. The guide garment, harness, and/or belt can be made in whole or part of spandex or an otherwise elastic material, such as to expand and conform to a torso when worn by the patient. Regardless of the structure, the applicator 50 ensures that the sensors 150 are properly and predictably placed at desired locations on the subject's body. In some embodiments, the confirmed use of applicator 50 places sensors with sufficient accuracy that additional location determination steps, such as scanning or sensor cross-signaling, are not included as part of the sensor location system 200.

Further, in some embodiments, the applicator 50 can also have one or more markings indicating where the sensors 150 should be placed and/or identifying locations where wires or waveguides from the data collection system 100 should be attached. The applicator 50 can also include integrated transmission paths, (e.g., wires, circuits, etc.) from the sensors 150 to reduce the number of free-standing wires connecting the sensors 150 to the data collection system 100. That is, the transmission path may be integrated into the applicator 50 to connect the sensors 150 to the data collection system 100. In the embodiment depicted in FIG. 4B, data concentration modules 152 for receiving leads coupled to the sensors 150 are attached to a waist belt 154. Each data concentration module 152 couples to eight leads from respective sensors 150 of a respective vertical column of sensors. That is, each column of sensors 150 is coupled to a corresponding data concentration module 152.

Each concentration module 152 includes an octopus cable having 8 leads for coupling to a respective column of sensors 150. Each concentration module 152 may further include an “RX pod” having a multiplexed eight-channel data acquisition chip with 24-bit ADC resolution specifically designed for ECG signals. The data acquisition chip may be coupled to an optoisolator and a serializer to transport the digitized signals to a field programmable gate array (“FPGA”). The FPGA is capable of receiving data at 8 kilo samples per second (“kSPS”). The high resolution ADCs isolate and extract the very weak cardiac signals of the PR Segment. The RX pod may be representative of the converter 110 of FIG. 3. The RX pod is configured to output the data representation of the cardiac signal to the collector 120 or another component of the system 10 for further data processing.

After applying the sensors 150 to the body, the scanner 60 may determine their locations in a two-dimensional (“2D”) or a three-dimensional (“3D”) space. The scanner 60 may be any device capable of imaging a body and generating data for a 3D image. For example, the scanner 60 may be an optical scanner, a laser scanner, a radio wave scanner, MRI imager, CT imager, fluoroscope, an electro-magnetic scanner, a smart phone, a tablet, and/or any other type of scanner capable of scanning a 3D object for generating data for a 3D model.

Regardless of the type of scanning device, the scanner 60 scans the subject's body and the applied sensors 150 to generate data for a 3D model of the subject indicating the locations of the applied sensors 150. The scanner 60 may pass over/around the subject and/or the subject may move with respect to scanner 60 to generate scan data of the desired portions of the subject's body (e.g., a patient's torso). The scanner 60 may also generate a 3D model based on the scan data. In some implementations, the sensor location system 200, the display system 400 and/or the CPU 40 may communicate with the scanner 60 and generate the 3D model based on the scan data.

Regardless of which component generates the 3D model based on the scan data from the scanner 60, the system 10 may identify the sensors 150 from the 3D model automatically using an algorithm. For example, a processor and/or memory (e.g., CPU 40) coupled to the scanner 60 may include a pattern-recognition algorithm and/or other algorithm configured to identify the locations of the sensors 150 in the 3D model. In some instances, two or more images of the subject can be captured by the scanner 60, such as from different angles, and the locations of the sensors 150 may be identified through the algorithm such that the locations of the sensors 150 are determined using stereoscopic calculations. The algorithm can include a pattern-recognition algorithm and/or other algorithm configured to identify the locations of the sensors 150, for example, machine learning, a neural network, and/or another artificial intelligence algorithm (“AI algorithm” herein). In some implementations, a user may indicate, highlight, or select the locations of the sensors 150 based on a 3D model generated from the scan data and in some implementations the algorithm may identify the 3D locations of the sensors based on a user's indication/selection.

In some embodiments, the sensors 150 are located by an algorithm that includes a geometric mesh algorithm. In such embodiments, two or more sensors 150 are fit (using a gradient descent or other optimizing or heuristic approach) to an ovoid-cylinder or a more anatomically realistic 3D torso model. In some embodiments, one or more sensors 150 can be identified as being at an anatomical location with a specific corresponding location on the torso model. The torso model can then be warped to best match the one or more distances between sensors 150. In some embodiments, the warping is constrained to preserve anatomical norms.

In some embodiments, a distance between two or more sensors 150 is measured physically (e.g., by a tape measure, ruler, and/or other measuring device) after the sensors 150 are placed. In some embodiments, a distance can be determined based on spacing guides built into the applicator 50. In some embodiments, a distance can be determined by having a first sensor 150 emit a signal of known strength (for example, in a sinusoidal pattern of known frequency and amplitude), having at least a second sensor 150 detect the strength of the signal received from the first sensor 150, and then calculating the distance between the sensors 150 based on the decrease in signal strength (e.g., based on the voltage drop using Ohm's law). This process can be repeated for every sensor 150 (in some embodiments, for a large number of pairs of sensors 150) such that all the locations of the sensors 150 are measured/computed. A distance calculation can be improved if the patient's torso skin impedance is measured first. In some embodiments, a skin impedance is calculated based on the drop in signal strength between two sensors 150 that are a known distance apart. Other embodiments locate the sensors using alternative methods.

In some embodiments, locations of the sensors 150 are determined using a 3D “standard” torso model. The “standard” torso model may be adjusted, warped, and/or otherwise fitted based on one or more measurements of the subject's/patient's torso to create an “adjusted torso model”. Sensors 150 are placed on the patient's torso-specific positions relative to anatomical reference points (in some embodiments, using the applicator 50 having one or more spacing guides). The 3D locations of the sensors 150 are then determined based on the locations of the same reference points on the adjusted torso model.

In embodiments in accordance herewith, relative locations of the sensors 150 can be refined as follows. Firstly, an initial estimate of locations is determined based on one of the methods described herein. Then each of the sensors 150, in turn, emits a signal, for example, a 10 Hz, 20 Hz, 50 Hz, 100 Hz, and/or 1000 Hz sinusoid. Next, non-signaling sensors 150 would calculate a center of electrical activity (“CEA”) of a signaling sensor 150 using single equivalent dipole (“SED”) algorithms. The calculated CEA then becomes a new estimate for the location of the signaling sensor 150. After completing this process for all sensors 150, the process can be repeated until a difference between a new estimate for the sensor's location and the previous estimate for the sensor's location is below a threshold (e.g., a predetermined threshold and/or a clinician-adjustable threshold.

Regardless of the method used for capturing and indicating sensor location data, the sensor location system 200 determines the location of the sensors 150 in relation to each other and the subject's body to verify proper sensor application and/or to enable accurate computation of SEDs. In some implementations, the sensor location system 200 may be omitted and the locations of the sensors 150 may be indicated by the clinician and uploaded or otherwise inputted into the system.

Referring now to FIG. 5, a flow diagram of a method 2 of collecting cardiac data by the data collection system 100 is illustrated. In step 2-1, the sensors 150 sense or collect electrical signals (e.g., analog signals) indicative of a heart's electrical activity during a cardiac cycle and/or of the location of the emitter and transmit the signals to the converter 110. In step 2-2, the converter 110 obtains the analog signals from the sensors 150 and converts the analog signals into digital signals (e.g., data). In step 2-3, the collector 120 aggregates or combines the digital data from the converter 110. In step 2-4, the collector 120 generates and/or outputs a raw data stream DS1 from the combined digital data. In some implementations, steps 2-3 and 2-4 may be combined into a single step.

In some embodiments, in step 2-3, the collector 120 combines partial data streams from all of the converters 110 to generate the raw data stream DS1 in step 2-4. In some embodiments, the converter 110 samples the signals (e.g., voltages) from the sensors 150 at, for example, 250 Hz, 500 Hz, 1000 Hz, 2000 Hz, 3000 Hz, 4000 Hz, 5000 Hz, 8000 Hz, 16000 Hz, 32000 Hz, or 64000 Hz. The collector 120 assembles data from the converter 110 (e.g., from all the converters of converter 110) into a single raw data stream DS1. In some embodiments, a signal is distributed among one or more converter components of the converter 110 to maintain coherent time stamps on data packets (e.g., despite the high sample rates). That is, the converter 110 may include one or more converter components that each convert data received from one or more sensors 150. The raw data stream DS1 contains digitized data from the one or more sensors 150, and can be embodied as a real-time buffered and/or unbuffered stream of data, and/or as a binary, ASCII, or otherwise-formatted file indexed by time and sensor identifier. The raw data stream DS1 can be stored in an electronic database stored in a memory, for example, a hard disk, SSD, and/or other device, that can be accessed by the data processing system 300. That is, the data processing system 300 may further include a memory, and/or may communicate with the memory 45 of the CPU 40. Any such database and/or memory suitable for use herein may be optimized for real-time storage and access.

At any time, a data stream DS1 stored in a database and/or in a memory 45 can be sampled, and data from it displayed graphically or otherwise (e.g., via one or more displays of a user interface 41 and/or the display system 400), using standard or custom software packages. This mid-stream data assessment can be used to assess the performance of the different steps in the overall data processing stream. In some embodiments, the values of individual center of electrical activity (“CEA”) coordinate across multiple sensors 150 and over time can be displayed. Data can be extracted in segments and stored in a memory 45 for backup and/or future analysis. For example, the data could be extracted in 0.1-minute, 1-minute, and/or 10-minute periods or segments.

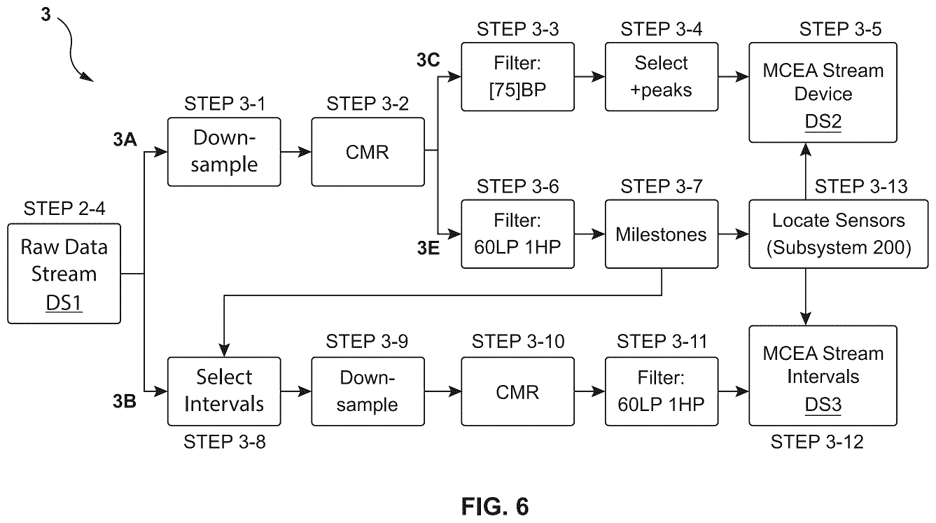

Referring now to FIG. 6, a flow chart of a method 3 for determining an MCEA of a subject's conduction pathway and/or an emitting device is illustrated, according to an embodiment. The method 3 includes transforming, via the data processing system 300, the raw data stream DS1 into MCEA data streams DS2 and/or DS3. The raw data stream DS1, representative of the signals collected by the sensors 150 in the method 2 of FIG. 4, is processed in several parallel paths (e.g., 3A, 3B, 3C and 3E), as shown in FIG. 6. Data transmitted along the paths can be passed as a real-time buffered and/or unbuffered data stream, and/or as binary, ASCII, or otherwise-formatted files indexed by time and sensor identifier. At each processing stage, the refined process data stream may be stored (e.g., in a memory 45) in a new file, a separate section of an existing file, in a single database indexed by the processing stage and the sample rate and/or in some other format. The steps of the method 3 need not be performed in the same order as shown in FIG. 6. For example, the order in which step 3-2 and step 3-3 or step 3-6 are performed can be reversed. Similarly, step 3-8 and/or step 3-9 can be performed after step 3-12. In some instances, one or more steps may be omitted.

In the depicted embodiment, the method 3 includes splitting or copying the raw data stream DS1 (from step 2-4 of FIG. 5) into two virtual streams that run along parallel first and second processing paths 3A and 3B, each of which can be processed at a different time resolution. For example, one stream may be processed at a low time resolution to analyze a whole cardiac cycles, or the P-Peak to R-Peak interval, and identify “good” heartbeats and segments thereof, (e.g., cardiac cycles or portions thereof having a desired morphology). The identified segments of selected “good” heartbeats are analyzed at high-time-resolution and/or high-data-resolution. This configuration enables efficient, real-time or near real-time (e.g., within 100 ms, 10 ms, 1 ms, or 0.1 ms) processing of high-time-resolution and/or high-data-resolution ECG data from specific parts of the cardiac cycle that are of interest (e.g., the PR segment). In addition to allowing hitherto-impossible detailed analysis of the low-intensity signal during the PR segment, this bifurcation also enables a high-fidelity analysis of deviations in the QT interval caused by downstream conduction defects, which in accordance with embodiments disclosed herein is a level of analysis that may be used to identify and locate conduction defects on the ventricular free walls, such as reentrant circuits, that lead to ventricular arrhythmias such as ventricular tachycardia.

In the first processing path 3A (e.g., a first data path), the data stream DS1 is first sampled down in a step 3-1. This allows for faster processing in subsequent steps. In some embodiments, data stream DS1 is down-sampled to 50 Hz, 100 Hz, 250 Hz, 500 Hz, 1000 Hz, or 2500 Hz. In various embodiments, the down-sampling can occur by a variety of techniques, such as decimation, resampling, interpolation, linear averaging, and/or curve fitting.

In a step 3-2, the data stream DS1 may undergo Common Mode Reduction (“CMR”) in which the average signal from the body for each time in the data stream is subtracted from the corresponding voltage value for each sensor 150 at that time. The CMR lessens common mode noise. In some embodiments, the average signal is the summation of the left arm, right arm, and left leg signals (referred to as Wilson's Central Terminal). In some embodiments, the average signal is derived from the aggregation of signals of a plurality of the sensors 150 attached to the torso and/or other body location(s).

In the step 3-2, or after the step 3-2, the data of the first processing path 3A is then bifurcated further into two data streams or paths (e.g., a third processing path 3C and a fourth processing path 3E). In a step 3-3, the third processing path 3C of the first processing path 3A can be reduced such as by using a narrow bandpass filter (e.g., included in the data processing system 300) to capture and isolate an electrical signal emanating from one or more emitting devices, for example the emitting device 90. If more than one emitting device 90 is deployed and being tracked, steps 3-3, 3-4, and 3-5 of FIG. 6 can be repeated to isolate the signals for each emitting device 90.

Using the now-isolated emitting device signal, each sensor 150 signal with positive peaks above a threshold voltage can be identified in a step 3-4. In some embodiments, this step can be performed by examining the signal for a period of time comprising a plurality of peaks and selecting those values that are in, for example, the top 1, 5, or 10% of voltages. All values in the stream for that time period may be replaced with the identified peak value and/or the average of the values above the threshold voltage. For example, the signal can comprise a 100 Hz sinusoid, the time period can be 0.1 s and contain 10 peaks. If, for one sensor 150, the maximum voltage during such a 0.1 s period is 10 mV, then all values above a threshold voltage of, for example, 9.5 mV can be averaged, and the signal from this sensor 150 for the entire 0.1 s period would be set to that average. In some embodiments, the signal from an emitting device 90 on each channel (e.g., each sensor 150 signal) is processed individually as follows: the system 10 (e.g., via an algorithm and/or the data processing system 300) reviews the signal moving forward to find the initial positive peak. Once the location of that peak is identified, the system 10 moves forward by whatever the period of the signal is and then searches for the next peak in an area around that region that may be 1, 5, 10 or 20% of the signal period. This process is repeated for the entire data set for all channels. System 10 then computes, via the data processing system 300 and/or CPU 40, the CEA for all data points, and/or for a small band of time at each peak. The SED at the peaks is then averaged for some time period, and that average can be used to track the MCEA of the emitting device 90 in real time. In some embodiments, a running average of a plurality of peaks is used to set the value of the signal from a sensor.

In a step 3-5, the data processing system 300 determines the CEA coordinates of the signal of the emitting device 90. Additionally, (when SED is used) the dipole vector for the signal of the emitting device 90, and thus, the orientation of the emitting device 90 may be determined. In particular, the processing system 300 determines the CEA coordinates for the signal of the emitting device 90 based on the determined MCEA of the emitting device 90 and the sensor 150 locations (determined by location system 200). This data can be timestamped and can become part of the processed data stream DS2 of the step 3-5. In instances where an emitter is not used, steps 3-3 through 3-5 may be omitted.

Meanwhile, in a step 3-6, the data generated in the step 3-2 traveling along the fourth processing path 3E is filtered through a 55 Hz low-pass filter (e.g., to eliminate power line noise) and a 1 Hz high-pass filter (e.g., to eliminate variations from slow movements) of the data processing system 300. For example, removing 60 Hz and its harmonics and maintaining the remainder leaves uncorrelated noise but allows that noise to be removed through cycle averaging. All filtering (e.g., steps 3-3, 3-6, 3-11) can be processed in segments large enough to minimize end effects but small enough to be efficient computationally. However, filtering too large a data stream may consume excessive computational resources and add latency. A filter with specific bounds, such as a 55 Hz low-pass filter, has different parameters for different sampling frequencies. For example, there can be one set of parameters for an 8 kHz sampling frequency and another for a 500 Hz sampling frequency. There are tradeoffs related to the “sharpness” of the filters implemented by the system 10. For filters implemented in software, where the border between passed and attenuated/rejected frequencies are sharper, faster, and/or harder, more parameters are needed by the filter software, hence the longer it takes to compute. In some embodiments, the data processing system 300 determines a parameter set that optimizes the tradeoff between noise and computational efficiency to achieve clinically valuable data in real-time. In some embodiments, additional filtering is applied to the data stream for the time periods when the system detects or is informed of the triggering of a pacing signal from an implanted pacing lead.

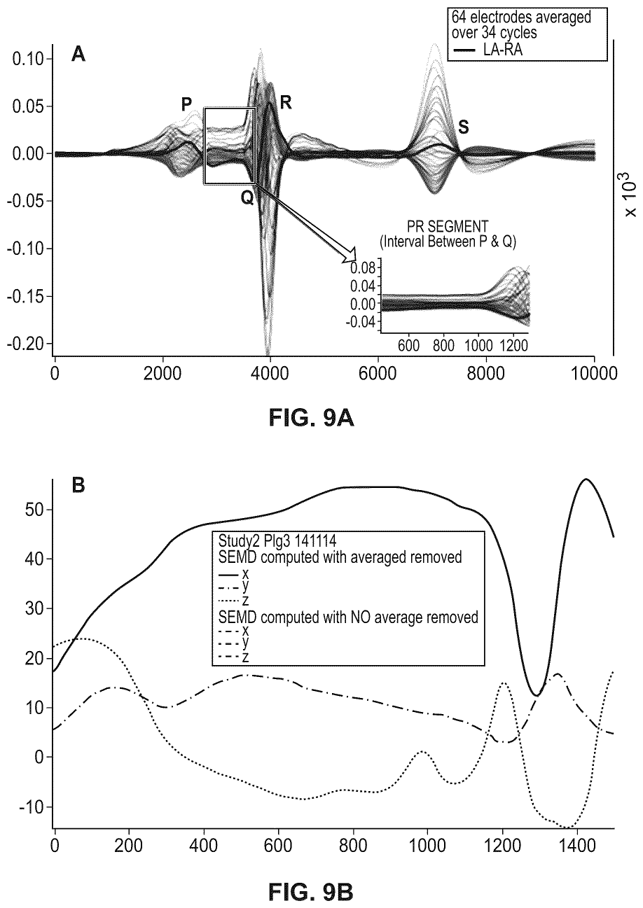

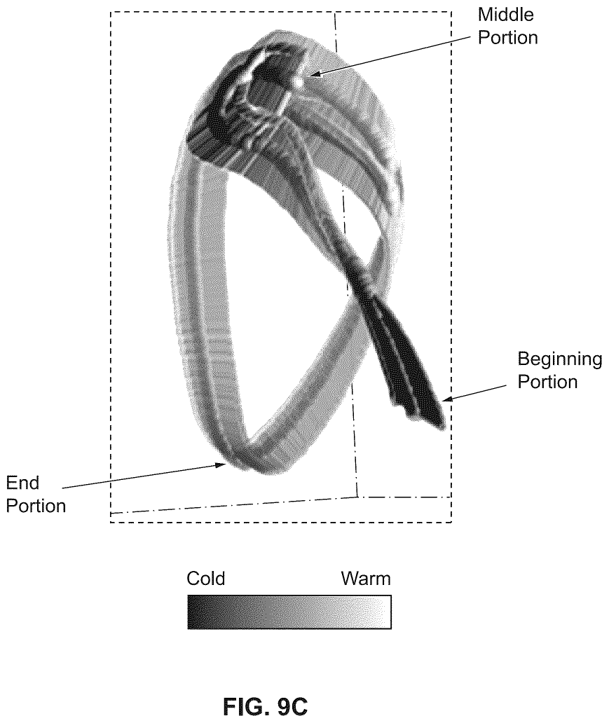

In a step 3-7, the data processing system 300 identifies the electrophysiological wave milestones for each heartbeat, using the filtered data from the step 3-6, to identify the relevant portions of the cardiac cycle (see FIG. 9A) for analysis. Next, a subset of the signals from the sensors 150 can be selected to calculate a differential ECG signal. In some embodiments, this subset of signals is from the sensors 150 placed on the extremities of a patient. In some embodiments, signals from a pair of sensors 150 applied to the torso is selected as the subset of signals. In some implementations, an algorithm, such as a signal smoothing algorithm, may be applied to the differential ECG signal stream to improve event detection. A pattern recognition algorithm can may be applied to identify a time stamp of each significant milestone (e.g., the peaks, beginnings, and ends of the P, Q, R, S, T) in the ECG cycle. In some implementations, the P-Peak to R-Peak interval may be identified as an ECG milestone. In some embodiments, the time stamp may be determined based on how well the SED algorithm predicts the pattern of voltages detected by the sensors: a small, final SED-fit error implies electrical activity on the heart is highly concentrated, as it is for the PR segment. The pattern recognition algorithm may be any known algorithm and/or software for physiological signal processing. These ECG milestones can be used by the data processing system 300 to timestamp a start/stop line for each heartbeat (which might be, for example, be set at a fixed time period before the P peak milestone). Steps 3-6 and 3-7 may be performed in parallel with steps 3-3 and 3-4. In some embodiments, the filtering could be performed in other ways.

In some embodiments, any irregular cardiac cycles/beats (such as premature ventricular contractions (“PVCs”), heartbeats where the software cannot identify all the milestones or where particular waves are unusually long or short, and/or other heartbeats that are aberrant in some other way) are identified and marked or labeled as “bad”. The method 3 may later exclude the “bad” heartbeat waves. In some implementations, the “bad” beats may be excluded from the data in the step 3-7 by the data processing system 300. In some embodiments, all the steps performed on the low resolution data are instead performed on high-data-resolution data.

In a step 3-8, in the second processing path 3B, high-time-resolution or high-data-resolution data from a subset of the data is extracted from the raw data stream DS1. The high-time-resolution data is determined by the data processing system 300 based on the milestone timestamps determined in Step 3-7. That is, heartbeat waves marked as “bad” are excluded from the data stream DS1, and only desirable heartbeat waves are selected. Further, a subset of data corresponding to the PR segment is selected as a desired segment. Because this step cannot be finalized until step 3-7 is complete, the processing of high-time-resolution or high-data-resolution data can lag behind the low-resolution processing in the fourth processing path 3E by up to the entire duration of the PR segment. When SED is used, this lag may be minimized by projecting when the desired segment (e.g., the PR segment) occurs based on data from prior heartbeats and by initiating SED calculations from the center of the forecasted PR segment. In some implementations, when the Q wave cannot be detected, located, or otherwise determined, the desired segment extends from the end of the P wave to the beginning of the R wave.

In a step 3-9 in the second processing path 3B, the data from the intervals selected in the step 3-8 is down-sampled for faster processing by the data processing system 300. The down-sampling in the step 3-9 may be substantially similar to the down-sampling in in the step 3-1 discussed above. However, in some embodiments, the data may be down-sampled to a lesser or greater extent than that in the step 3-1. In some implementations, the step 3-9 may be skipped or omitted because only a relatively small portion of the data corresponding to the desired segment (e.g., the PR segment) is processed. Because inter-milestone (e.g., segment) durations may vary from heartbeat to heartbeat, the down-sampling can involve resampling (for example, by curve fitting and/or weighted averaging) so that the number of samples in the interval remains constant across heartbeats.

In some embodiments, for the desired segment (e.g., PR segment) the system 10 (e.g., via the data processing system 300) resamples the ECG data in the segment for every “good” cardiac cycle so that there are the same number of data points in the segment in every cycle. This resampling can be performed because the duration of each segment varies between cycles and the number of actual data points collected during the desired segment may vary from cycle to cycle. When the desired segment extends from the end of the P wave to the beginning of the R wave, the system 10 uses the first section of the desired segment, for example the first 25%, 30%, 50%, 70%, or 90% of the interval, to analyze the PR segment.

In a step 3-10, CMR is applied to the processed data stream output from the step 3-9 (or from the step 3-8 in implementations where the step 3-9 is omitted) in a similar manner to the CMR applied in the step 3-2. In the step 3-11, the processed data from the step 3-10 is filtered, for example using a 55 Hz low-pass filter (which eliminates power line noise and automatically excludes any signal from an emitting device, if used, that is at a higher frequency such as 75 Hz, 90 Hz, 120 Hz, 200 Hz, 300 Hz, or 800 Hz) and a 1 Hz high-pass filter to eliminate variations from slow movements (e.g., due to respiration, heartbeats, and manipulation of the subject, internal tissue, surgical instruments, and/or the emitting device 90).

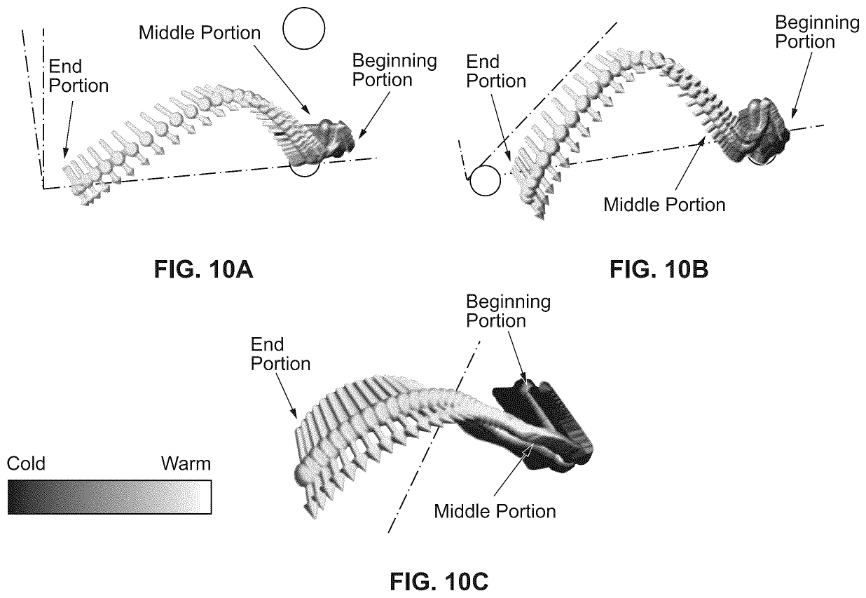

In a step 3-12, the CEA coordinates and dipole vector for the heart's electrical signal at each sampled time point during the selected interval is determined based on the resulting processed data stream from the step 3-11 and the sensor locations data determined from the step 3-13, discussed in detail below. A space curve (see FIGS. 9C, and 10A-10C) representing the MCEA data stream DS3 is generated based on the collection of CEA coordinates. That is, the CEA coordinates are plotted over time and correspond to an electrical impulse, or MCEA, as it propagates along the cardiac conduction pathway (e.g., the AV node, the His Bundle, the RBB, the LBB, and the Purkinje fibers) during the PR segment. The plotted CEA coordinates generate the space curve that represents the MCEA data. Thus, the MCEA data corresponds to the locations of the AV node, the His Bundle, the RBB, the LBB, and the Purkinje fibers. Consequently, the space curve plots are also graphical representations of a section of the cardiac conduction pathway (e.g., the AV node, the His Bundle, the RBB, the LBB, and the Purkinje fibers). In some embodiments, data on the CEA fit error is added to the data stream DS3. This data can be timestamped and can become part of the processed data stream DS3.

Because CEA calculations require knowing the relative 3D locations of the sensors 150, this location data must be fed into those calculations at a step 3-13. In the step 3-13, the locations of the sensors 150 are determined, as described above, as part of the sensor location system 200. In some embodiments, the system 10 (e.g., the sensor location system 200, the data collection system 100, etc.) maintains a list of “bad” sensors 150 and excludes and/or down-weighs those automatically from the CEA calculations. For example, a “bad” sensor might be one identified by the system 10 as having no signal or a low signal-to-noise ratio.

In some embodiments, CEA forward-propagation calculations are adjusted by weighting sensor data based on the field propagation impedance expected between the heart and the sensor 150. This impedance-based weighting can be determined by the system 10 (e.g., via the CPU 40) using the pure dipole signal emitted by the emitting device 90. For example, the weights for the sensors 150 are adjusted until the predicted sensor recorded voltages reflect the observed voltages.

Alternatively, or additionally, torso impedance can be measured across different torso chords by having a first sensor 150 positioned on one side of the body emit a signal while having a second sensor 150 positioned on the opposite side of the body receive that signal, and calculating the drop in signal strength. In some embodiments, the voltage drop across a chord that transits the heart can be used to calculate weights for those sensors 150 on either side of the chord. Alternatively or additionally, data on voltage drops across all chords can be used to generate an impedance map of the torso. Such a map can be based on a 3D anatomic model of volumes and locations of the organs in which resistance estimates of individual organs are adjusted to fit the drop-vector data. In some embodiments, the 3D anatomic model is adjusted for expected abdominal fat deposits, such as adjustments performed (e.g., via an algorithm) based on gender, BMI, and/or other factors.

In some embodiments, the CEA is calculated using SED analysis; however, other techniques, such as iTSI, can be used. SED seeks to describe an electrical dipole (location and orientation/moment) that would produce an electric field that would, in turn, generate voltages on the sensors 150 placed at specific points on the surface of the torso of the subject or patient. The SED can be inferred from sensed voltages using inverse algorithms, such as those described below. The SED may be plotted over time to show a SEMD corresponding to an MCEA along the cardiac conduction pathway. Accordingly, an SED analysis of the sensor data may be used to calculate a CEA as it travels along the cardiac conduction pathway.

Standard calculation of propagated voltages assumes that the inside of the body has uniform conductivity. Due to the presence of bones and organs, the conductivity of the torso is not uniform. While standard calculations do not precisely predict the surface voltages, an inverse algorithm can find a “best fit” of the SED model to the sensed or observed data. Any distortion in the SED locations due to the inhomogeneity of the torso varies continuously as the CEA in the cardiac conduction pathway moves. Additionally, distortions are small (e.g., within 100 micrometers to a few millimeters) for SEDs determined for multiple entities in close proximity to each other, such as the cardiac conduction pathway in the heart and an emitting device 90 disposed within the subject's heart. Accordingly, the non-uniform conductivity of the body may be, but need not be, accounted for in the SED model to usefully represent the cardiac conduction pathway and/or the emitting device 90 and enable navigation/guidance of the emitting device 90 to the cardiac conduction pathway.

Using the techniques herein, the system 10 generates locations of the space curve of the SEMD in “SED-space” (a slightly distorted version of real space) representing the MCEA data stream DS3 and also generates the emitting device(s)′ dipole(s) location(s), orientation(s), and movement representing the MCEA data stream DS2 (if an emitting device 90 is present). Using this information, the cardiac conduction pathway (e.g., the AV node, the His Bundle, the LBB, the RBB, etc.) and the emitting device 90 (if present) may be displayed by the system 10 so clinicians can clearly navigate the emitting device 90 to a desired target within the heart of a patient. That is, for example, the system 10 generates a visual representation of a device's SED with respect to the heart's SEMD (indicative of the MCEA) such that the device may be guided to the cardiac conduction pathway. To further enable this navigation, the system 10 may overlay the SED-space with an image of the heart so that the catheter may be guided to the cardiac conduction pathway. Consequently, a pacing lead proximate to the emitting device 90 may be guided to and collocated with the heart's cardiac conduction pathway to achieve conduction system pacing. In some embodiments, the system 10 is both a display and a navigation system.

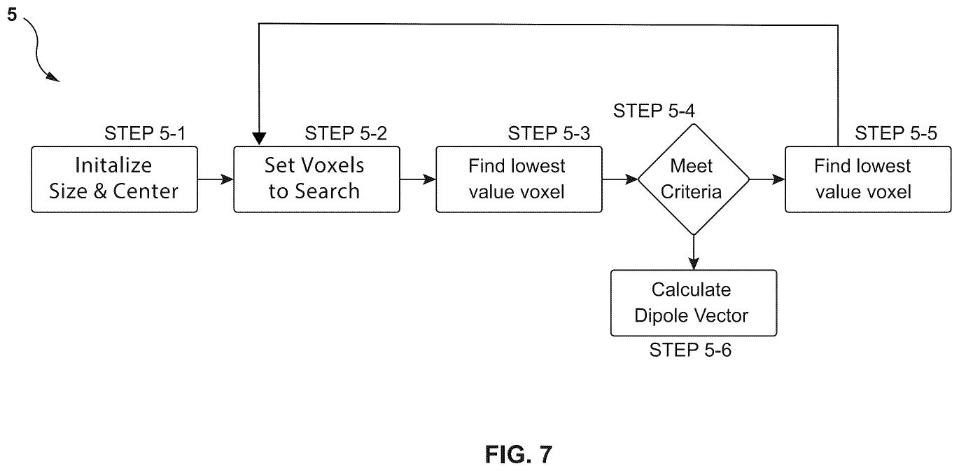

There are several methods for calculating the SED parameters for a time point (e.g., steps 3-5 and 3-12). In some embodiments, this calculation is performed by the system 10 through a gradient descent algorithm that optimizes the error with which the SED fits the sensor data. In some embodiments, the calculation is done as a non-linear approximation, through an approximate forward operator, training a neural network, machine learning, AI, and/or in some other manner. In some embodiments, the calculation is done through a voxel grid search such as the one described below in reference to FIG. 7.

Referring now to FIG. 7, a flow chart of a method 5 for determining a location of an SED is illustrated, according to an embodiment. A voxel grid search performed by the system 10 can be iterative, thereby searching more carefully with each pass of an area where the best SED fit was found. In a step 5-1, the search is initialized by determining and setting the search area/volume center and size. In some embodiments, the initial search volume is centered around the patient's torso and/or heart and is large enough to encompass the entire torso. In some embodiments, the search volume is a cube. In some embodiments, the center and size of the search volume are defined using the location coordinates for the sensors 150 placed on the torso. In some embodiments, the center is offset toward the left ventral side of the chest of the patient. In some embodiments, the search volume is limited to 1, 2, or 3 times the volume of the heart of the patient.

In some embodiments, the search is centered around an SED solution in a recent time period or cardiac cycle. For example, the search for the SED in a time period is centered around the location of the SED for the previous time period, around a projection of where the next SED should be based on the locations of the SEDs in some number of recent time periods, or around the average position of the SED at the same relative time in one or more previous cardiac cycles, or some combination of the above. In some embodiments, the search volume is limited to the area with a 95% or 99% chance of containing the SED based on previous SEDs.

In a step 5-2, a search set comprising a voxel volume to be searched is determined (e.g., by data processing system 300) based on sensitivity factor S. The search area/volume is divided into S³ voxels, creating an S×S×S voxel cube. Any voxels outside the search volume, such as the torso volume or the heart volume, are deleted from the search set. In some embodiments, the search volume is defined by the location coordinates of the sensors 150 placed on the torso. In some embodiments, the search volume is defined by an elliptical cylinder fit to the coordinates of the sensors 150. In embodiments where voxels that fall outside the heart's volume are deleted, the volume of the heart can be determined by the system 10 based on imaging data (e.g., captured by an imager 80 that may be, for example, a fluoroscope, MRI or CT), and/or based on a standard anatomic model adjusted based on the size of the patient's torso as defined by the locations of the sensors 150 on the torso.

In a step 5-3, a SED-fit error value is calculated by the system 10 for each voxel remaining in the search set to find the voxel with the best SED fit. The system 10 (e.g., the data processing system 300) calculates the difference between a calculated electric field strength of a dipole located in a center of a particular voxel for each sensor 150 and the actual field strength measured by each sensor 150 for that particular voxel. Individual voxel errors are aggregated (e.g., by a weighted sum). The SED-fit error can then be calculated in various ways.

In a step 5-4, the SED-fit error and/or the size of the voxel S are compared to a criteria. The criteria includes a SED-fit error of, for example, less than 0.01%, 0.1%, 1%, 2%, 5%, or 10% of the weighted sum of the squared voltages at each sensor and the voxel size may be, for example, less than 1 mm, less than 0.25 mm, or less than 0.01 mm. If the criteria is not met, the method continues to a step 5-5. If the criteria is met, the method continues to a step 5-6.

In the step 5-5, the center of the lowest error value voxel is selected as the new center of the search cube. The method 5 returns to the step 5-2 and iteratively repeats until the SED-fit error and voxel size meet the criteria in the step 5-4. When the method returns to the step 5-2, the size of the new search cube is equal to that of the 27 voxels containing and surrounding the previously determined lowest value voxel. In some embodiments, the search is restarted using a larger initial volume if the SED-fit error is not small enough once the voxel size has reached the terminating value. In some implementations, the criteria may be the number of times the voxel search space has been refined. For example, the voxel search space criteria may be a predetermined number of times the voxel search space is refined. In some instances, the criteria is met after two, three, four, five, or more iterations of voxel search space refinements.

Returning to the step 5-4, if the error is small enough or the size of the voxel is small enough, the criteria is met, the search concludes, the SED coordinates are set as the voxel's center, and the method proceeds to the step 5-6. In some embodiments, the terminating voxel size would be less than 3 mm, less than 1 mm, less than 0.25 mm, or less than 0.01 mm. In the step 5-6, the dipole moment vector is calculated from the processed data stream DS3 data from the sensors 150 on the torso.

In some embodiments, the system 10 (e.g., via data processing system 300) reviews each new CEA calculation for temporal continuity, and rejects solutions that suggest discontinuous movement in the space curve. In other embodiments, the resulting space curve of CEA points is smoothed, and each CEA value modified, enabling the CEA calculation to consider a more extensive data set, including those in temporally proximate locations, thereby reducing the standard error of the CEA estimate.

In some embodiments, CEA data is averaged across two or more cardiac cycles to better estimate the average conduction pathway. However, because the heart physically moves and stretches as it beats, and because this movement may vary from heartbeat to heartbeat, the location of the space curve of the conduction path with respect to the torso may also vary from heartbeat to heartbeat. Therefore, to develop a more accurate estimate of the conduction pathway, the movement of the heart may be detected, for example, by monitoring respiration, heart noises, an echocardiogram, fluoroscopy, and/or signals from an accelerometer on the lead-placing emitting device 90, and to use that information to adjust the CEA space curve for each cardiac cycle. In some embodiments, CEA data taken during the QRS interval is used to estimate the movement of the heart.

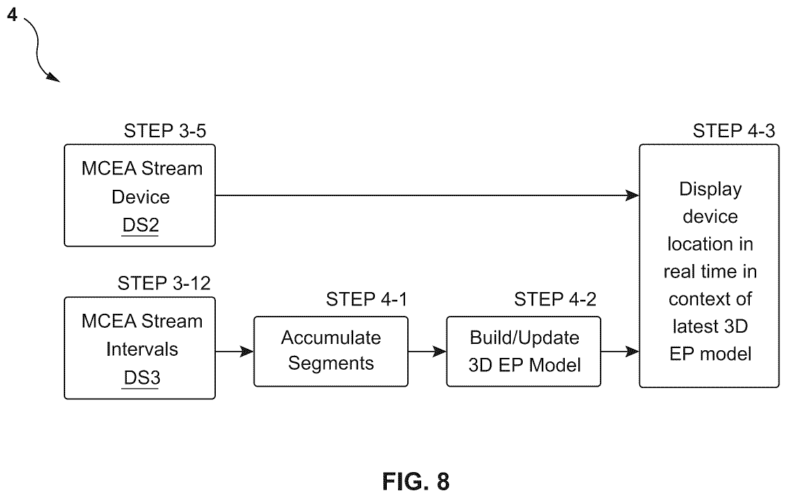

Referring now to FIG. 8, a flow chart of a method 4 for displaying data is illustrated, according to one or more embodiments. The display system 400 receives the MCEA data streams DS2 and/or DS3 produced by the data processing system 300 and generates or produces a graphical 3D model of the information for displaying on a GUI or display. The 3D model may include a graphical representation of the heart (either a generic heart shape or of the patient's specific heart derived from imaging data) overlayed with one or more space-curves generated from the MCEA data. As noted above, the MCEA may be used to generate a CEA space curve over time; the space curve can be a SEMD space curve, generated using SED analysis. The space-curves are indicative of the cardiac conduction pathways (e.g., AV node, His Bundle, LBB, RBB, etc.) and may be fitted to a graphical representation of the subject's heart. In some implementations, the 3D model may further include a graphical representation of the location and orientation of the emitting device 90 (if present).

As depicted in FIG. 8, the method 4 includes accumulating a plurality of selected intervals from the stream DS3 of MCEA points, e.g., MCEA location and, if SED is used, the dipole vector for each time sample, in a step 4-1. In some embodiments, any MCEA sequences that are deemed to be non-representative based on a criteria (e.g., ECG waveforms that diverge from the normal waveform of the patient's heartbeat and/or otherwise aberrant cardiac waveforms due to prolonged or foreshortened waves or segments, missing, extra, or malformed waves, arrhythmias, improper heartbeat, signal interference, etc.) are excluded (e.g., via the data processing system 300 and/or the display system 400). In some embodiments, the selected intervals are PR segments and the MCEA points correspond to an electrical signal propagating along the AV node, His Bundle, LBB and RBB. However, any desired interval or desired segment of the ECG wave may be selected to detect a desired conduction pathway.

In a step 4-2, the datasets for the collected intervals can be used by the system 10 (e.g., via the data processing system 300 and/or the display system 400) to develop and/or update a 3D model of the electrophysiology (for example, of the cardiac conduction system) of the heart. In some embodiments, the 3D model is a simple averaging of the MCEA locations/dipole vectors across multiple heartbeats. For example, the 3D model may be represented as SEMD space curves (such as those illustrated in FIGS. 9C and 10A-10C) that can be averaged considering variations in heart motion, as described above. That is, the 3D model may account for distortions due to motion of the heart. In addition, since the cardiac conduction pathway (“CCP”) may, in some patients, be slightly diffuse and the CEA may vary from beat to beat, the averaging may give a more accurate representation of the center of the CCP. In some embodiments, all or some portion of the data is fitted to a linear, parametric, and/or another model, and standard errors are calculated. For example, a line or planar curve might be fitted to the PR segment MCEA, representing the AV node, the His Bundle, the LBB, and/or the RBB. In some embodiments, where a planar curve is fit to the PR segment, the curve's containing plane can be used as an estimate of the location of the heart's septum. In some embodiments, the plane of the septum, as determined by the emitting device as described below, can be used to inform or constrain the curve fit to the MCEA. In some embodiments, the accuracy (e.g., the standard error) of the curve fit is calculated once or dynamically. In some embodiments, this accuracy may be displayed graphically. For example, a probability distribution of the location of the CCP may be displayed on an image of the septum. In some implementations, the MCEA space curves may be overlayed with a graphical representation of the heart. The graphical representation of the heart may be based on data from fluoroscopy, MRI, CT, and/or other imaging techniques. In some implementations, the range of values the MCEA might have (e.g., its standard error) is overlayed with the graphical representation of the space curve, for example with error bars or a transparent error cloud.

In some embodiments, the system 10 can be configured to collect data when the emitting device 90 is moved around the inside of the heart while its CEAs are determined. This data can be used (e.g., by the data processing system 300 and/or the display system 400) to create a SEMD-space map of the location of different parts of the heart, such as the septum, apex, and free-wall of a ventricle or an atrium. The set of CEA points inside the heart can be used by the system 10 to interpolate and map a surface for the endocardium. In some embodiments, the system 10 uses this technique to locate the surface of the septum, which, in turn, is used to inform and/or constrain the 3D model into which the MCEA space curve is fit. For example, the emitting device can be navigated using fluoroscopy to the estimated location of the mid septum, then moved around in all directions while gradually decreasing depth toward the valves. Such a technique avoids the trabecular of the lower septum and the chordae of the valves near the upper septum. Then an algorithm, such as a shrink-wrap manifold mesh generation algorithm, can be used to identify a surface that encompasses the cloud-map of where the emitting device has been. The septum portion of that surface can be isolated based on its orientation. The surface can also be used to properly position, scale, or warp the heart model that is overlayed.

In some embodiments, the fit is modified by the system 10 (e.g., via the data processing system 300 and/or the display system 400) to account for the physical distortion of the heart as it beats. In some embodiments, data on MCEA fit quality or inter-interval errors is used to refine the model further. Once this model is created, it can be updated by the system 10 as additional data is collected. The model can be stored in memory and/or reset during a procedure (e.g., run before and after therapy is applied), for example to determine the impact of pacing and/or other therapies on the model.

In a step 4-3, once this 3D model of the patient's heart is created by the system 10, the SED location and dipole vector of the emitting device 90 can be displayed with the 3D model via the display system 400 in real-time. Accordingly, a clinician may locate the emitting device 90 and its orientation with respect to the septum and/or the cardiac conduction system model in real time and then move the emitting device 90 until it reaches the desired position and orientation (e.g., perpendicular to the plane of the septum) with respect to the septum and/or the cardiac conduction system model, and thus the patient's heart. For example, in some embodiments, the dipole vector of the emitting device 90 can be used to determine the orientation of emitting device 90, which can then be used to adjust the angle with which the emitting device 90 inserts an instrument, such as a pacing lead, into the target (e.g., septum). In some embodiments, the CEA location of the emitting device 90 relative to the septum can be used to calculate the depth with which an instrument is inserted.

The system 10 includes a GUI configured to provide many features designed to assist the user (e.g., a clinician). The GUI may be a component of the display system 400, or a component of the CPU 40 (e.g., of the user interface 41) in communication with one or more of the data collection system 100, the sensor location system 200, the data processing system 300, and the display system 400. For example, the GUI can display standard ECG signals and/or vector electrocardiograms, the CEA data in with a representation of the heart (e.g., a transparent and/or cut-away 3D model of the heart, a 3D model of just the septum, and/or an image of the heart provided by an imager). The GUI may be configured to rotate and/or translate the image of the CEA data and heart representations based on inputs from the user, such as to achieve a desired viewing angle. The GUI could also be configured to display more than one viewing angle simultaneously. The GUI can also display an illustrative graphic of a lead and the emitting device 90, as oriented in space with respect to the representation of the heart, the representation of the septum, and/or the model of the MCEA space curves of the PR segment.

In some embodiments, the GUI is configured to display data based on the electrical fields detected by the one or more electrodes on the emitting device 90. This data could, for example, indicate the proximity of the emitting device to electrically active heart tissue or could, based on the shape of the ECG pattern detected, suggest where in the heart the emitting device 90 is (e.g., in an atria or in a ventricle).

In some embodiments, the GUI includes one or more flat screen displays and/or tablet computers that are connected by wires or wirelessly to one or more other components of the system 10. Controls for managing the user interface 41 and/or modifying the parameters used to process the sensor data and MCEA data can be via a touch screen and/or a separate user input device like a keyboard, mouse, joystick, trackpad, and/or custom-designed control or haptic control device. The touch screen may also be a display presenting the MCEA and graphical representation of the heart or may be a separate device.

In some embodiments, the GUI can be configured to allow control of the emitting device 90. For example, controls on the GUI can manage (e.g., turn on or off, and/or adjust the frequency or amplitude of) the signal emitted from the emitting device 90. In some embodiments, this control is accomplished through a module of the user interface 41 that is attached to wires on the proximal end of the emitting device 90 or to the signal generator 91 or is accomplished through a wireless connection (e.g., Bluetooth, WiFi, or ZigBee) to the proximal end of the emitting device 90 or to the signal generator 91. In some embodiments, the user interface 41 can issue commands to a robotic controller connected to the emitting device 90, where the controller can actuate the movement of the emitting device 90 within the heart.

The GUI can be configured to display the location of the HIS-Purkinje system (inclusive of bundle branches) within the septum, such as by using only the near proximate information (direction, magnitude, or angle) of the HIS-Purkinje system collected by the MCEA data or using the cardiac conduction system 3D model described above.

The user interface 41 can be a standalone display or integrated (e.g., co-registered) into another image of the heart created from another imaging modality, such as from an imager 80 that may be a fluoroscope, CT imager, MRI imager, and/or other imaging device.

There are multiple similar and alternate uses for the system 10 of the present disclosure. For example, space curves representative of MCEA data collected by the system 10 for part or all of the cardiac cycles can be used to: study variability in heart function (contractility, cardiac output, and the like); optimize left ventricular lead placement; optimize pacemaker timing intervals (A-V intervals, V-V intervals, and the like); aid in the understanding of heart conduction speeds throughout the cardiac cycle; diagnose arrhythmias and/or locate sources of arrhythmias (such as reentrant circuits); and/or aid in the understanding of the impact of myocardial scars on heart conduction. MCEA space curves determined by the system 10 before and after an intervention (such as placing a pacing lead) can be used by the system 10 (e.g., via an algorithm) to assess the impact of the intervention on broken or dysfunctional conduction pathways. For example, the space curves could be assessed to determine whether conduction system pacing (“CSP”) has been achieved. Individual cardiac cycle or averaged SEMD space curves can be compared to standard curves for healthy patients or patients with specific heart pathologies. The system 10 can include one or more algorithms (e.g., derived by way of machine learning) that are applied to the SEMD space curves to identify particular heart function pathologies. These and other types of clinical analyses of the data produced by the system 10 can be used for: diagnosing patients; determining whether the patient is a good candidate for a particular type of pacing (such as conduction system pacing); verifying the effectiveness of an intervention post-procedure; and/or monitoring a patient in the months and years after an intervention.

For example, if the ECG and/or SEMD values meet a criteria (e.g., a desired morphology, desired values, etc.), a particular type of pacing (i.e., a pacing strategy) may be selected for the patient. Alternatively, if the ECG and/or the SEMD values do not meet the criteria, the patient may not be a good candidate for pacing. After a conduction system pacing procedure, the ECG and/or SEMD values may be compared to a second criteria (e.g., desired morphology, desired values, etc.) to determine the effectiveness of the intervention. For example, the ECG and/or the SEMD data may be compared to a desired morphology. The lower the deviation from the desire morphology to the measured morphology, the greater the predicted effectiveness of the pacing.

Now referring to FIG. 9A, extracted valid ECG data from 64 separate electrodes (e.g., sensors 150) that is averaged over 34 heartbeats with a focus on the PR segment, is depicted. FIG. 9B is a graph of X. Y. and Z SEMD values calculated from the extracted valid ECG data for the PR segment, in FIG. 9A. Although ECG signals during the PR segment are relatively weak, different combinations of 8 kHz, 24-bit sampling, appropriate noise filtering, and/or averaging across heartbeats, as discussed above, allowed for zooming in to the PR segment to achieve high time-resolution SEMD during the passage of the electrical signal from the AV node to His Bundle, the LBB and RBB. From this data, space curves across multiple heartbeats and zoomed in on the PR segment may be modeled for a variety of mammals.

FIG. 9C depicts an example SEMD space curve illustrating the calculated orientation and magnitude for each SED point in a P-peak to R-peak interval within a 3D volume. That is, a cooler (blue to green) beginning portion of the space curve represents the orientations and magnitudes of SED points at the beginning of the P-peak to R-peak interval while a warming (red) middle portion of the space curve represents the orientations and magnitudes of SED points during the middle of the P-peak to R-peak interval. Finally, a warmer (orange to yellow) end portion of the space curve represents the orientations and magnitudes of SED points at the end of the P-peak to R-peak interval. A desired segment (e.g., a PR segment) of the P-peak to R-peak interval may be isolated from the SEMD space curve of FIG. 9C as depicted, for example, in FIGS. 10A to 10C described below. The space curve of FIG. 9C may be representative of the ECG wave propagating through the AV node, the His Bundle, the LBB, the RBB, and the Purkinje fibers during the P-peak to R-peak interval. Accordingly, using the techniques presented herein, the cardiac conduction pathway, including the AV node, the His Bundle, the LBB, and the RBB may be detected and mapped in real-time.

SEMD space curves shown in FIGS. 10A-10C are illustrative of PR segments for three different sets of data representative of an MCEA propagating through the AV node, the His Bundle, the LBB and the RBB. 3D locations of the MCEA as it propagates along the cardiac conduction pathway is represented by an SED moving in 3D space and is displayed as a plurality of arrows or vectors over a period of time in FIGS. 10A-10C. Each arrow of the SEMD space curve is indicative of a 3D location and direction of an SED at a particular time instance. For instance, a cooler (blue) portion of the SEMD space curve is indicative of the SED at the beginning of the desired segment (e.g., the PR segment). A warming (red to orange) middle portion of the space curve is indicative of the SED of the MCEA at the middle portion of the desired segment. Finally, a warmer (yellow) end portion of the space curve is indicative of the SED at the end portion of the desired segment.

In each of these SEMD space curves, during the desired segment (e.g., the PR segment), significant changes in the moment of the SED, the direction of movement of the SED, and the speed of movement of the SED are evidenced by a spacing and direction of travel of the plurality of arrows/vectors in FIGS. 10A-10C. In particular, during these selected segments, the magnitude of the moment was small (suggesting a wave front involving only a small number of polarized cells at any given time) and the SED (represented by an arrow at a particular location along the cardiac conduction pathway) first paused then moved rapidly in a single direction (as indicated by the locations, spacings, and colors of the arrows/vectors). These changes corresponded well to the expected location and behavior of the electrical signals that would be produced while the electrical wave of cardiac conduction was “delayed” in the AV node then progressed down the HIS bundle and the LBB and the RBB to the Purkinje fibers. By contrast, the SEMD during the QRS complex, which is known to be associated with the broad cell activation wave associated with ventricular contraction, had a much larger dipole moment and a SEMD that moved in the opposite direction from that during the PR segment. Similarly, the dipole moment size and movement during the P wave was consistent with the articular activation known to happen during that period. Because the cellular discharge is highly localized in the PR segment. SED locations trace out the physical path from the AV node into the His Bundle, the LBB, the RBB and the Purkinje fibers.

Accordingly, using the techniques described herein, different combinations of high-time-resolution (e.g., 8 kHz) and high data-resolution (e.g., 24-bit) sampling of the electric fields produced during the relatively weak PR segment, or desired segment between the end of the P wave to the beginning of the R wave (when the beginning of the Q wave cannot be detected or otherwise determined), with appropriate noise filtering, and/or averaging across heartbeats achieves high resolution SEMD data (representative of an MCEA) during the passage of the electrical signal from the AV node to the His bundle to the LBB and the RBB. This SEMD data may be plotted and displayed as a space-curve to a clinician in real time. Moreover, using the techniques presented herein the location and orientation of dipole-emitting catheter or electrode may be distinctly presented in the same frame of reference as the space curve of the SEMD data to enable a clinician to navigate the catheter to targeted locations in the cardiac conduction system. That is, the MCEA data may be overlayed, in real time, with an image of the heart. The catheter may also be visible within the image, thereby providing a 3D view of at least a tip of the catheter, the MCEA data on the cardiac conduction system, and the heart. The clinician may then navigate the catheter to the desired location in the heart using the 3D images. The catheter may then apply a therapy (e.g., ablation, attach pacing leads or other electrode, etc.) to the desired location of the heart

In some implementations, the system may be configured to provide feedback on whether the electrode was placed in a location that captures the conduction system (i.e., results in conduction system pacing) and/or to confirm the specific physiologic location (His Bundle, LBB, RBB, etc.) of the electrode. The system may also indicate whether the electrode was placed outside of the conduction system pathway. In some embodiments, the system may confirm conduction system capture by making measurements with pacing and without pacing by the electrode (i.e., pacing vs. intrinsic). Such measurements may include QRS interval, QRS width, QRS axis, potential to QRS intervals, transitions in QRS morphology, paced latency intervals, positive/negative appearance of the QRS in different leads of a 12-lead ECG (for example, positive QRS in lead II), and additional 12-lead ECG measures (for example, R wave peak time in leads V5 or V6). Such measures may be directly captured or synthesized from other electrodes. In some embodiments, the system may confirm conduction system pacing based on the morphology of the MCEA space curve with and without pacing.

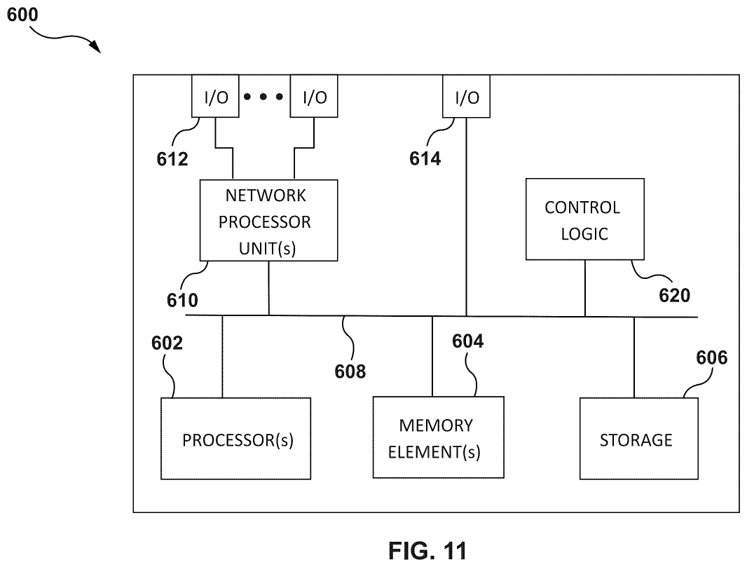

Now referring to FIG. 11, a hardware block diagram of a computing device 600 that may perform functions associated with operations discussed herein in connection with the techniques depicted in FIGS. 3-10C is depicted. In various embodiments, a computing device or apparatus, such as a computing device 600 or any combination of computing devices 600, may be configured as any entity/entities as discussed for the techniques depicted in connection with FIG. 3 in order to perform operations of the various components (e.g., the system 10, the data collection system 100, the sensor location system 200, the data processing system 300, the display system 400, the CPU 40, the scanner 60, and/or the imager 80) discussed herein.

In at least one embodiment, the computing device 600 may be any apparatus that may include one or more processor(s) 602, one or more memory element(s) 604, a storage 606, a bus 608, one or more network processor unit(s) 610 interconnected with one or more network input/output (I/O) interface(s) 612, one or more I/O interface(s) 614, and a control logic 620. In various embodiments, instructions associated with logic for the computing device 600 can overlap in any manner and are not limited to the specific allocation of instructions and/or operations described herein.

In at least one embodiment, the processor(s) 602 is/are at least one hardware processor configured to execute various tasks, operations and/or functions for a computing device 600 as described herein according to software and/or instructions configured for the computing device 600. Processor(s) 602 (e.g., a hardware processor) can execute any type of instructions associated with data to achieve the operations detailed herein. In one example, the processor(s) 602 can transform an element or an article (e.g., data, information) from one state or thing to another state or thing. Any of potential processing elements, microprocessors, digital signal processor, controllers, systems, CPU, GPU, device, and/or machines described herein can be construed as being encompassed within the broad term “processor”. In some embodiments, the processor 602 utilizes a graphic processing unit (GPU) or other parallel processing architecture to simultaneously process multiple threads, for example to simultaneously evaluate the SED-error for multiple voxels or average values over different intervals.

In at least one embodiment, the memory element(s) 604 and/or the storage 606 is/are configured to store data, information, software, and/or instructions associated with the computing device 600, and/or logic configured for the memory element(s) 604 and/or storage 606. For example, any logic described herein (e.g., a control logic 620) can, in various embodiments, be stored for the computing device 600 using any combination of the memory element(s) 604 and/or storage 606. Note that in some embodiments, the storage 606 can be consolidated with the memory element(s) 604 (or vice versa), or can overlap/exist in any other suitable manner.

In at least one embodiment, the bus 608 can be configured as an interface that enables one or more elements of the computing device 600 to communicate in order to exchange information and/or data. The bus 608 can be implemented with any architecture designed for passing control, data and/or information between processor(s), memory elements/storage, peripheral devices, and/or any other hardware and/or software components that may be configured for the computing device 600. In at least one embodiment, the bus 608 may be implemented as a fast kernel-hosted interconnect, potentially using shared memory between processes (e.g., logic), which can enable efficient communication paths between the processes.

In various embodiments, the network processor unit(s) 610 may enable communication between the computing device 600 and other systems, entities, etc., via a network I/O interface(s) 612 (wired and/or wireless) to facilitate operations discussed for various embodiments described herein. In various embodiments, the network processor unit(s) 610 can be configured as a combination of hardware and/or software, such as one or more Ethernet driver(s) and/or controller(s) or interface cards, Fiber Channel (e.g., optical) driver(s) and/or controller(s), wireless receivers/transmitters/transceivers, baseband processor(s)/modem(s), and/or other similar network interface driver(s) and/or controller(s) now known or hereafter developed to enable communications between the computing device 600 and other systems, entities, etc. to facilitate operations for various embodiments described herein. In various embodiments, network I/O interface(s) 612 can be configured as one or more Ethernet port(s), Fiber Channel ports, any other I/O port(s), and/or antenna(s)/antenna array(s) now known or hereafter developed. Thus, the network processor unit(s) 610 and/or network I/O interface(s) 612 may include suitable interfaces for receiving, transmitting, and/or otherwise communicating data and/or information with other devices and/or systems.

The I/O interface(s) 614 allow for input and output of data and/or information with other entities that may be connected to the computing device 600. For example, the I/O interface(s) 614 may provide a connection to external devices such as a keyboard, keypad, a touch screen, and/or any other suitable input and/or output device now known or hereafter developed. In some instances, external devices can also include portable computer readable (non-transitory) storage media such as database systems, thumb drives, portable optical or magnetic disks, and memory cards. In still some instances, external devices can be a mechanism to display data to a user, such as, for example, a computer monitor, a display screen, or the like.

In various embodiments, the control logic 620 can include instructions that, when executed, cause the processor(s) 602 to perform operations, which can include, but not be limited to, providing overall control operations of computing device; interacting with other entities, systems, etc. described herein; maintaining and/or interacting with stored data, information, parameters, etc. (e.g., memory element(s), storage, data structures, databases, tables, etc.); combinations thereof; and/or the like to facilitate various operations for embodiments described herein.

The programs described herein (e.g., a control logic 620) may be identified based upon application(s) for which they are implemented in a specific embodiment. However, it should be appreciated that any particular program nomenclature herein is used merely for convenience; thus, embodiments herein should not be limited to use(s) solely described in any specific application(s) identified and/or implied by such nomenclature.

In various embodiments, any entity or apparatus as described herein may store data/information in any suitable volatile and/or non-volatile memory item (e.g., magnetic hard disk drive, solid state hard drive, semiconductor storage device, random access memory (RAM), read only memory (ROM), erasable programmable read only memory (EPROM), application specific integrated circuit (ASIC), etc.), software, logic (fixed logic, hardware logic, programmable logic, analog logic, digital logic), hardware, and/or in any other suitable component, device, element, and/or object as may be appropriate. Any of the memory items discussed herein should be construed as being encompassed within the broad term ‘memory element’. Data/information being tracked and/or sent to one or more entities as discussed herein could be provided in any database, table, register, list, cache, storage, and/or storage structure: all of which can be referenced at any suitable timeframe. Any such storage options may also be included within the broad term ‘memory element’ as used herein.

Note that in certain example implementations, operations as set forth herein may be implemented by logic encoded in one or more tangible media that is capable of storing instructions and/or digital information and may be inclusive of non-transitory tangible media and/or non-transitory computer readable storage media (e.g., embedded logic provided in: an ASIC, digital signal processing (DSP) instructions, software [potentially inclusive of object code and source code], etc.) for execution by one or more processor(s), and/or other similar machine, etc. Generally, memory element(s) 604 and/or storage 606 can store data, software, code, instructions (e.g., processor instructions), logic, parameters, combinations thereof, and/or the like used for operations described herein. This includes the memory element(s) 604 and/or storage 606 being able to store data, software, code, instructions (e.g., processor instructions), logic, parameters, combinations thereof, or the like that are executed to carry out operations in accordance with teachings of the present disclosure.

In some instances, software of the present embodiments may be available via a non-transitory computer useable medium (e.g., magnetic or optical mediums, magneto-optic mediums, CD-ROM, DVD, memory devices, etc.) of a stationary or portable program product apparatus, downloadable file(s), file wrapper(s), object(s), package(s), container(s), and/or the like. In some instances, non-transitory computer readable storage media may also be removable. For example, a removable hard drive may be used for memory/storage in some implementations. Other examples may include optical and magnetic disks, thumb drives, and smart cards that can be inserted and/or otherwise connected to a computing device for transfer onto another computer readable storage medium.

To the extent that embodiments presented herein relate to the storage of data, the embodiments may employ any number of any conventional or other databases, data stores or storage structures (e.g., files, databases, data structures, data or other repositories, etc.) to store information.

Note that in this specification, references to various features (e.g., elements, structures, nodes, modules, components, engines, logic, steps, operations, functions, characteristics, etc.) included in ‘one embodiment’, ‘example embodiment’, ‘an embodiment’, ‘another embodiment’, ‘certain embodiments’, ‘some embodiments’, ‘various embodiments’, ‘other embodiments’, ‘alternative embodiment’, and the like are intended to mean that any such features are included in one or more embodiments of the present disclosure, but may or may not necessarily be combined in the same embodiments. Note also that a module, engine, client, controller, function, logic or the like as used herein in this specification, can be inclusive of an executable file comprising instructions that can be understood and processed on a server, computer, processor, machine, compute node, combinations thereof, or the like and may further include library modules loaded during execution, object files, system files, hardware logic, software logic, or any other executable modules.

It is also noted that the operations and steps described with reference to the preceding figures illustrate only some of the possible scenarios that may be executed by one or more entities discussed herein. Some of these operations may be deleted or removed where appropriate, or these steps may be modified or changed considerably without departing from the scope of the presented concepts. In addition, the timing and sequence of these operations may be altered considerably and still achieve the results taught in this disclosure. The preceding operational flows have been offered for purposes of example and discussion. Substantial flexibility is provided by the embodiments in that any suitable arrangements, chronologies, configurations, and timing mechanisms may be provided without departing from the teachings of the discussed concepts.

Example 1. A system for mapping at least a portion of a cardiac conduction pathway in a patient, the system comprising: a plurality of sensors; a data collection system for collecting sensor data from the plurality of sensors; a data processing system for calculating a center of electrical activity (CEA) data from the sensor data; and a display system for presenting a three-dimensional (3D) model of a portion of the cardiac conduction pathway based on the calculated CEA data.

Example 2. The system of example 1, wherein the CEA data is determined by calculating a single equivalent dipole (SED).

Example 3. The system of example 1 or 2, wherein the 3D model of the portion the cardiac conduction pathway is displayed with respect to images of a gross anatomy of a heart.

Example 4. The system of any one of examples 1 to 3, wherein the plurality of sensors detect a signal propagating through segments of the cardiac conduction pathway during a PR segment of a cardiac cycle.

Example 5. The system of any one of examples 1 to 4, wherein the display system displays the portion of the cardiac conduction pathway located between an atrioventricular (AV) node and Purkinje fibers of a heart.

Example 6. The system of any one of examples 1 to 5, wherein the data collecting system comprises a digital converter for generating high resolution data from very low voltage signals sensed by the plurality of sensors.

Example 7. The system of example 6, wherein the voltage signals are below 0.1 mV.

Example 8. The system of any one of examples 1 to 7, wherein the data processing system enhances the sensor data via one or more of a low pass filter; a high pass filter; common mode reduction; and/or differentially weighting data from different sensors of the plurality of sensors.

Example 9. The system of any one of examples 1 to 8, further comprising a sensor location system for identifying a location of each sensor of the plurality of sensors in a 3D space.

Example 10. The system of example 9, wherein the sensor location system comprises a scanner, a CT machine, and/or an MRI machine configured to generate a 3D image.

Example 11. The system of example 10, wherein a machine learning algorithm trained to identify sensors in a 3D image identifies and locates the plurality of sensors in the 3D image generated by the sensor location system.

Example 12. The system of example 9, further comprising a garment and/or a harness for receiving the plurality of sensors.

Example 13. The system of any one of examples 1 to 12, wherein the data collection system is configured to indicate to a user whether a particular sensor of the plurality of sensors is improperly positioned and/or malfunctioning.

Example 14. The system of any one of examples 1 to 13, wherein the data collection system is configured to cause one or more sensors of the plurality of sensors to transmit one or more signals.

Example 15. The system of any one of examples 1 to 14, wherein the system is configured to filter the sensor data by applying a different broad band pass filter and/or narrow band pass filter to selected frequencies.

Example 16. The system of example 15, wherein the selected frequencies are between about 0.5 and 55 Hz or between about 65 and 300 Hz.

Example 17. The system of example 15, wherein the data processing system is configured to remove CEA data corresponding to timepoints for which a voltage at a particular timepoint of the timepoints is below a threshold value.

Example 18. The system of any one of examples 1 to 17, wherein each sensor of the plurality of sensors is weighted when determining a CEA based on a voltage-drop across at least one chord between sensors of the plurality of sensors.

Example 19. The system of any one of examples 1 to 18, wherein a location of a portion of the cardiac conduction pathway is determined by combining CEA data from multiple cardiac cycles.

Example 20. The system of example 19, wherein the combined CEA data from multiple cardiac cycles accounts for discrepancies due to motion of a heart during each cardiac cycle.

Example 21. The system of example 19, wherein the combined CEA data comprises combining the CEA data from the multiple cardiac cycles into a best fit a model.

Example 22. The system of example 21, wherein the system is further configured to determine a location of a septum of a patient's heart using data collected from an emitting device.

Example 23. The system of example 22, wherein the location of the septum constrains the best fit model of the cardiac conduction pathway.

Example 24. The system of example 22, wherein the system is used for navigating a catheter to a target on the septum.

Example 25. The system of example 21 wherein a probability distribution for the location of the cardiac conduction pathway is graphically displayed on an image of the septum.

Example 26. The system of any one of examples 1 to 25, further comprising an emitting device, wherein the data processing system is configured to determine a location of the emitting device relative to the location of a portion of the cardiac conduction pathway.

Example 27. The system of example 26, further comprising a control device connected to a proximal end of the emitting device.

Example 28. The system of example 27, wherein the control device activates and/or controls a signal emitted by the emitting device.

Example 29. The system of example 27, wherein the control device controls movement of the emitting device.

Example 30. The system of any one of examples 1 to 29, further comprising an emitting device, wherein the data processing system is configured to determine an orientation of the emitting device relative to a location of the portion of the cardiac conduction pathway.

Example 31. The system of any one of examples 1 to 30, wherein a location of the portion of the cardiac conduction pathway is determined before and after a therapy is applied to the patient.

Example 32. The system of any one of examples 1 to 31, wherein the data processing system enhances the sensor data by selecting a filter based on whether or not an implanted pacing lead has recently delivered a pacing signal.

Example 33. The system of any one of examples 1 to 32 where the system is also used for navigating a catheter to a target in the heart.

Example 34. The system of any one of examples 1 to 33, wherein the system can also be used to develop a pacing strategy for a patient.

Example 35. The system of any one of examples 1 to 34, wherein the system can also be used to determine whether conduction system pacing has been achieved.

Example 36. A method of mapping at least a portion of a cardiac conduction pathway comprising: sensing, via sensors, signals indicative of cardiac electrical signals propagating through the cardiac conduction pathway; combining the signals from each sensor, via a data collection system, to generate a first data stream; identifying, via a data processing system, a waveform from the data stream via a low resolution sampling of the first data stream; sampling, via the data processing system, a segment of the identified waveform of the first data stream at a high-resolution to generate a second data stream; determining, via the data processing system, center of electrical activity (CEA) data based on the second data stream; and generating, via a display system, a three-dimensional (3D) model of the CEA data in real time, wherein the 3D model is indicative of the cardiac conduction pathway.

Example 37. The method of example 36, wherein determining the CEA data comprises performing single equivalent dipole (SED) analysis of the second data stream.

Example 38. The method of example 36 or 37, further comprising generating a real time image of a heart and overlaying the 3D model with reference to the real time image of the heart.

Example 39. The method of any one of examples 36 to 38, wherein the identified waveform is an electrocardiogram comprising a P wave, QRS complex and T wave.

Example 40. The method of example 39, wherein the segment of the identified waveform is a PR segment of the identified waveform.

Example 41. The method of any one of examples 36 to 40, further comprising determining locations of the sensors with respect to a heart of a subject.

Example 42. The method of example 41, wherein determining locations of the sensors comprises scanning the subject and the sensors, wherein the sensors are disposed on a body of the subject.

Example 43. The method of example 41, wherein determining CEA data is further based on the determined locations of the sensors.

Example 44. The method of any one of examples 36 to 43, further comprising sensing, via the sensors, a single dipole signal from an emitting device disposed within a heart of a subject.

Example 45. The method of example 44, further comprising determining, via the processing system, a location and orientation of the emitting device using SED analysis.

Example 46. The method of example 45, further comprising generating, via the display system, a 3D representation of the emitting device with respect to the 3D model indicative of the cardiac conduction pathway, wherein the 3D representation indicates a location and orientation of the emitting device in real time.

Example 47. The method of example 46, further comprising navigating the emitting device so that it is proximate to a desired portion of the cardiac conduction pathway based on the 3D representation.

Example 48. The method of example 46, further comprising detecting an electrode has been placed so as to capture proximal to the cardiac conduction pathway.

Each example embodiment disclosed herein has been included to present one or more different features. However, all disclosed example embodiments are designed to work together as part of a single larger system or method. This disclosure explicitly envisions compound embodiments that combine multiple previously discussed features in different example embodiments into a single system or method.

While the invention has been illustrated and described in detail and with reference to specific embodiments thereof, it is nevertheless not intended to be limited to the details shown, since it will be apparent that various modifications and structural changes may be made therein without departing from the scope of the inventions and within the scope and range of equivalents of the claims. In addition, various features from one of the embodiments may be incorporated into another of the embodiments. Accordingly, it is appropriate that the appended claims be construed broadly and in a manner consistent with the scope of the disclosure as set forth in the following claims.

Reference may be made to the spatial relationships between various components and to the spatial orientation of various aspects of components as depicted in the attached drawings. However, as will be recognized by those skilled in the art after a complete reading of the present disclosure, the devices, components, members, apparatuses, etc. described herein may be positioned in any desired orientation. Thus, the use of terms such as “above,” “below,” “upper,” “lower,” “top,” “bottom,” or other similar terms to describe a spatial relationship between various components or to describe the spatial orientation of aspects of such components, should be understood to describe a relative relationship between the components or a spatial orientation of aspects of such components, respectively, as the components described herein may be oriented in any desired direction. When used to describe a range of dimensions and/or other characteristics (e.g., time, pressure, temperature, distance, etc.) of an element, operations, conditions, etc., the phrase “between X and Y” represents a range that includes X and Y.

For example, it is to be understood that terms such as “left,” “right,” “top,” “bottom,” “front,” “rear,” “side,” “height,” “length,” “width,” “upper,” “lower,” “interior,” “exterior,” “inner,” “outer” and the like as may be used herein, merely describe points of reference and do not limit the present invention to any particular orientation or configuration. Further, the term “exemplary” is used herein to describe an example or illustration. Any embodiment described herein as exemplary is not to be construed as a preferred or advantageous embodiment, but rather as one example or illustration of a possible embodiment.

Further, the present disclosure may repeat reference numerals and/or letters in the various examples. This repetition is for the purpose of simplicity and clarity and does not in itself dictate a relationship between the various embodiments and/or configurations discussed.

Similarly, when used herein, the term “comprises” and its derivations (such as “comprising,” etc.) should not be understood in an excluding sense, that is, these terms should not be interpreted as excluding the possibility that what is described and defined may include further elements, steps, etc. Meanwhile, when used herein, the term “approximately” and terms of its family (such as “approximate,” etc.) should be understood as indicating values very near to those which accompany the aforementioned term. That is to say, a deviation within reasonable limits from an exact value should be accepted, because a skilled person in the art will understand that such a deviation from the values indicated is inevitable due to measurement inaccuracies, etc. The same applies to the terms “about” and “around” and “substantially”.

As used herein, unless expressly stated to the contrary, use of the phrase “at least one of,” “one or more of,” “and/or,” variations thereof, or the like are open-ended expressions that are both conjunctive and disjunctive in operation for any and all possible combination of the associated listed items. For example, each of the expressions “at least one of X, Y and Z,” “at least one of X, Y or Z,” “one or more of X, Y and Z,” “one or more of X, Y or Z” and “X, Y and/or Z” can mean any of the following: 1) X, but not Y and not Z; 2) Y, but not X and not Z; 3) Z, but not X and not Y; 4) X and Y, but not Z; 5) X and Z, but not Y; 6) Y and Z, but not X; or 7) X, Y, and Z.

Additionally, unless expressly stated to the contrary, the terms “first,” “second,” “third,” etc., are intended to distinguish the particular nouns they modify (e.g., element, condition, node, outlet, inlet, valve, module, activity, operation, etc.). Unless expressly stated to the contrary, the use of these terms is not intended to indicate any type of order, rank, importance, temporal sequence, or hierarchy of the modified noun. For example, “first X” and “second X” are intended to designate two “X” elements that are not necessarily limited by any order, rank, importance, temporal sequence, or hierarchy of the two elements. Further as referred to herein, “at least one of” and “one or more of” can be represented using the “(s)” nomenclature (e.g., one or more element(s)).

1. A system for mapping at least a portion of a cardiac conduction pathway in a patient, the system comprising:
a plurality of sensors;
a data collection system for collecting sensor data from the plurality of sensors;
a data processing system for calculating a center of electrical activity (CEA) data from the sensor data; and
a display system for presenting a three-dimensional (3D) model of a portion of the cardiac conduction pathway based on the calculated CEA data.

2.-3. (canceled)

4. The system of claim 1, wherein the plurality of sensors detect a signal propagating through segments of the cardiac conduction pathway during a PR segment of a cardiac cycle.

5. The system of claim 1, wherein the display system displays the portion of the cardiac conduction pathway located between an atrioventricular (AV) node and Purkinje fibers of a heart.

6. The system of claim 1, wherein the data collecting system comprises a digital converter for generating high resolution data from very low voltage signals sensed by the plurality of sensors.

7. The system of claim 6, wherein the voltage signals are below 0.1 mV.

8.-20. (canceled)

21. The system of claim 1, wherein a location of a portion of the cardiac conduction pathway is determined by combining the CEA data from multiple cardiac cycles into a best fit a model.

22. The system of claim 21, wherein the system is further configured to determine a location of a septum of a patient's heart using data collected from an emitting device.

23. The system of claim 22, wherein the location of the septum constrains the best fit model of the cardiac conduction pathway.

24. The system of claim 22, wherein the system is used for navigating a catheter to a target on the septum.

25. The system of claim 22, wherein a probability distribution for the location of the cardiac conduction pathway is graphically displayed on an image of the septum.

26.-30. (canceled)

31. The system of claim 1, wherein a location of the portion of the cardiac conduction pathway is determined before and after a therapy is applied to the patient.

32.-35. (canceled)

36. A method of mapping at least a portion of a cardiac conduction pathway comprising:
sensing, via sensors, signals indicative of cardiac electrical signals propagating through the cardiac conduction pathway;
combining the signals from each sensor, via a data collection system, to generate a first data stream;
identifying, via a data processing system, a waveform from the data stream via a low resolution sampling of the first data stream;
sampling, via the data processing system, a segment of the identified waveform of the first data stream at a high-resolution to generate a second data stream;
determining, via the data processing system, center of electrical activity (CEA) data based on the second data stream; and
generating, via a display system, a three-dimensional (3D) model of the CEA data in real time, wherein the 3D model is indicative of the cardiac conduction pathway.

37.-39. (canceled)

40. The method of claim 36, wherein the segment of the identified waveform is a PR segment of the identified waveform.

41. The method of claim 36, further comprising determining locations of the sensors with respect to a heart of a subject by scanning the subject and the sensors, wherein the sensors are disposed on a body of the subject.

42. (canceled)

43. The method of claim 41, wherein determining CEA data is further based on the determined locations of the sensors.

44. The method of claim 36, further comprising sensing, via the sensors, a single dipole signal from an emitting device disposed within a heart of a subject.

45. The method of claim 44, further comprising determining, via the processing system, a location and orientation of the emitting device using SED analysis.

46. The method of claim 45, further comprising generating, via the display system, a 3D representation of the emitting device with respect to the 3D model indicative of the cardiac conduction pathway, wherein the 3D representation indicates a location and orientation of the emitting device in real time.

47. The method of claim 46, further comprising navigating the emitting device so that it is proximate to a desired portion of the cardiac conduction pathway based on the 3D representation.

48. (canceled)

49. The system of claim 1, further comprising an emitting device, wherein the data processing system is configured to determine a location of the emitting device relative to the location of a portion of the cardiac conduction pathway.
